SEQUENCE LISTING (111 sequences)

SEQ ID NO: 1 - DNA, 2676 bp, Solanum lycopersicum
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 2 - DNA, 2677 bp, Artificial Sequence
  SYNTHETIC POLYNUCLEOTIDE
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttccgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 3 - DNA, 2677 bp, Artificial Sequence
  SYNTHETIC POLYNUCLEOTIDE
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagttctttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 4 - DNA, 2677 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 5 - DNA, 2677 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaactttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 6 - DNA, 2679 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatttagtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 7 - DNA, 2656 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 8 - DNA, 2675 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcattaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 9 - DNA, 2658 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttatgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 10 - DNA, 2671 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatataatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 11 - DNA, 2666 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 12 - DNA, 2673 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 13 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaattttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 14 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcatttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 15 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttccttttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 16 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcatttttttctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 17 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaataccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 18 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaaccccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 19 - DNA, 2676 bp, Artificial Sequence
  synthetic polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatgtgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 20 - DNA, 2676 bp, Artificial Sequence
  synthetic polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattgttttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 21 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaatttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 22 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagatatgaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 23 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcggagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 24 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcaaagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 25 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgcagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 26 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtggggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 27 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtagtaaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 28 - DNA, 2676 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcctagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 29 - DNA, 2678 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaattttttttctttcatttaacagttttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaaccttttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 30 - DNA, 2627 bp, Artificial Sequence
  Synthetic Polynucleotide
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagttatttaagttgtgtttggttatgaatatgaattagagttgtttttttcctttttttctcaattatttcgagtaaacttttttttttctttaaagaattgaaattttatgagttttaactatcgaaaaaaagcgaaaaagatcaaatcccctctaatagtttttattaatcaattaaatacattttcaatagtgactatgacgacataatatttatataatatgtgattgttttatcaaaaaattttaaaatttaattttcacgtctttcttttcttttgaaacgtcattttttatatttagtaccttttagatccaatatctatctatggatagacgttgcgaagtactttttgttattttcaattattaggcacaaataattgaatctagcacctcttgtatgtacaaaattttaaactgtagcaataaataaatatattttttaatttttttttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 31 - DNA, 2626 bp, Solanum lycopersicum
cgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaattttttttctttcatttaacagttctttttataatattttatttcgaaggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagttatttaagttgtgtttggttatgaatatgaattagagttgtttttttcctttttttctcaattatttcgagtaaacttttttttttctttaaagaattgaaattttatgagttttaactatcgaaaaaaagcgaaaaagatcaaataccctctaatagtttttattaatcaattaaatacattttcaatagtgactatgacgacataatatttatataatatgtgattgttttatcaaaaaattttaaaatttaattttcacgtctttcttttcttttgaaacgtcattttttatatttagtaccttttagatccaatatctatctatggatagacgttgcgaagtactttttgttattttcaattattaggcacaaataattgaatctagcacctcttgtatgtacaaaattttaaactgtagcaataaataaatatattttttaatttttttttttttttgtctgagcagatagtgatatcgaatggccaagactcgaggagattatgccagacgtggtgattgatgatgaagatatgaacacaaatttcatattgaattgtttcgaagaagaagtaacgagcaataatgcggggaatagttattcatgtatcgaggaaggtagtaaaaagatatcaagcgacgatgaaaaaatcaaattattaatggattggcaagataatgatgagttagtatggccaacgttaccatgggaattagaaacggatatagttcctagttggccacaatgggacgatactgacactaacttacttcaaaattgcaccaatgataataattatgaagaagcaacaacaatggaaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaa

SEQ ID NO: 32 - DNA, 1248 bp, Solanum lycopersicum
atgggaagaacaccttgttgtgaaaaagtgggcatcaagagaggcagatggactgcagaagaagatcaaattctcactaattatattatttctaatggagaaggctcttggaggtcgttacctaaaaatgccggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaaaaaaaaaaaaaaaaaaaaaaaaaaaaa

SEQ ID NO: 33 - protein, 338 aa, Solanum lycopersicum
  misc_feature (255)..(255): Xaa can be any naturally occurring amino acid
MGRTPCCEKVGIKRGRWTAEEDQILTNYIISNGEGSWRSLPKNAGLLRCGKSCRLRWINYLRSDLKRGNITSQEEDIIIKLHATLGNRWSLIAEHLSGRTDNEIKNYWNSHLSRKVDSLRIPSDEKLPKAVVDLAKKGIPKPIKKSSISRPKNKKSNLLEKEALCCTNMPACDSAMELMQEDLAKIEVPNSWAGPIEAKGSLSSDSDIEWPRLEEIMPDVVIDDEDKNTNFILNCFREEVTSNNVGNSYSCIEEXNKKISSDDEKIKLLMDWQDNDELVWPTLPWELETDIVPSWPQWDDTDTNLLQNCTNDNNNYEEATTMEINNQNHSTIVSWLLS

SEQ ID NO: 34 - DNA, 1152 bp, Solanum lycopersicum
cacaacaaagccaaaaaaacttccattatccttctctcaattggtatatcacacttgcaaaaaccaaaaaaaataaaggtatagtttcttaaatactcttatttgtctaaatgggaaggtcaccttgttgtgagaaggcacatacaaacaaaggagcatggactaaagaagaagatgaaagactaatttcttacattagagctcatggtgaaggttgttggaggtctcttcctaaagctgctggacttcttcgatgcggtaaaagttgtcgtctccgatggattaattacttaagacctgaccttaaacgtggtaactttactgaagaagaagatgaactcattatcaaactccatagcctccttggaaacaagtggtcgcttatagcaggaagattaccaggaagaacagataacgagataaaaaactattggaacacacatataagacgaaagctcttgagtcgaggtattgatccaacaacacatagatcaatcaatgatcctactacaataccaaaagttacaacgattacttttgctgctgctcatgaaaatattaaagatattgatcaacaagatgagatgataaatatcaaagctgaattcgttgaaacaagcaaagaatcagataataatgaaataattcaagaaaagtcatcatcatgtcttcctgacttaaatcttgaactcagaattagtcctccacatcatcaacaactcgatcatcatcgtcatcatcaacgatcaagctctttatgttttacatgtagtttgggaattcaaaatagtaaagattgcagttgtggaagtgaaagtaatggaaatggatggagtaataatatggtaagtatgaacattatggctggttatgactttttgggcttgaagactaatggtcttttggactatagaactttggaaactaagtgattcgattcgattcgaatttatgcataaatcatcttaatttgatgtatatgtagaaaagaaaaagagtttgaggatattatttcttctagctaataattttctcatggttgtaaactttgcaatatagtaattacatttaattcaaagcagtaaaaatatagcactaatgattctatgaagataatttttctttttcctctaaaaaaaaaaaaaaaaaaaa

SEQ ID NO: 35 - protein, 273 aa, Solanum lycopersicum
MGRSPCCEKAHTNKGAWTKEEDERLISYIRAHGEGCWRSLPKAAGLLRCGKSCRLRWINYLRPDLKRGNFTEEEDELIIKLHSLLGNKWSLIAGRLPGRTDNEIKNYWNTHIRRKLLSRGIDPTTHRSINDPTTIPKVTTITFAAAHENIKDIDQQDEMINIKAEFVETSKESDNNEIIQEKSSSCLPDLNLELRISPPHHQQLDHHRHHQRSSSLCFTCSLGIQNSKDCSCGSESNGNGWSNNMVSMNIMAGYDFLGLKTNGLLDYRTLETK

SEQ ID NO: 36 - DNA, 1282 bp, Solanum lycopersicum
cacaacaaagccaaaaaaacttccattatccttctctcaattggtatatcacacttgcaaaaaccaaaaaaaataaaggtatagtttcttaaatactcttatttgtctaaatgggaaggtcaccttgttgtgagaaggcacatacaaacaaaggagcatggactaaagaagaagatgaaagactaatttcttacattagagctcatggtgaaggttgttggaggtctcttcctaaagctgctggacttcttcgatgcggtaaaagttgtcgtctccgatggattaattacttaagacctgaccttaaacgtggtaactttactgaagaagaagatgaactcattatcaaactccatagcctccttggaaacaagtatgtttaacatttctatcttattttattttgtcctactagttaaatcgttaacgagatgagacagattcaggatttaaatttgacctgactcaaggtggttatatggatcactagtagtaattatgtttttttgtttccttatgataggtggtcgcttatagcaggaagattaccaggaagaacagataacgagataaaaaactattggaacacacatataagacgaaagctcttgagtcgaggtattgatccaacaacacatagatcaatcaatgatcctactacaataccaaaagttacaacgattacttttgctgctgctcatgaaaatattaaagatattgatcaacaagatgagatgataaatatcaaagctgaattcgttgaaacaagcaaagaatcagataataatgaaataattcaagaaaagtcatcatcatgtcttcctgacttaaatcttgaactcagaattagtcctccacatcatcaacaactcgatcatcatcgtcatcatcaacgatcaagctctttatgttttacatgtagtttgggaattcaaaatagtaaagattgcagttgtggaagtgaaagtaatggaaatggatggagtaataatatggtaagtatgaacattatggctggttatgactttttgggcttgaagactaatggtcttttggactatagaactttggaaactaagtgattcgattcgawtcgaatttatgcataaatcatcttaatttgatgtatatgtagaaaagaaaaagagtttgaggatattatttcttctagctaataattttctcatggttgtaaactttgcaatatagtaattacatttaattcaaagcagtaaaaatatagcactaatgattctatgaagataatttttctttttcctct

SEQ ID NO: 37 - DNA, 1865 bp, Solanum lycopersicum
ctcaaatccttgaatactcaaaggacaaaaataaaaaaaattcaaagtatgggacgttcaccttgttgtgaaaaagcacatacaaataaaggagcatggactaaagaagaagaccaacgcctcatcaattatatacgtgctcatggggaaggttgctggcgttctcttcctaaagctgcaggtattcattgtcgaaaaattatattgtatatatattaaaaaaaatattttgaaatatctcgctcgttatacatatattgaaaaattaaacttattaaatttaaattaagaaattaagaggatctaataatttttggatctactgccacaaaaaaaaaagattatggatgtggataaaagattttgagacttttttttgtaaaatattgaattatttttaaaaatattttaggattgtcaagatgtggaaagagttgcagattgagatggataaattatttaaggcctgatctcaaaagagggaattttacagaagaagaagatgaattgataatcaaacttcatagtttgcttggaaacaagtatgtatatatgatctttgttattttaatttatttttttggttttacttattataagttatatattcctagtatcataaaatgtgtgaaaattggagtagaaagtcaaaattgttttcttttttgtttggctaatttgggaccacattaagaagttataaaaacaagatctaaattgttctaagagaaaaaagtggttatagtagtaaatttttacaagtgggaaagacttctctgtttcttgaaataccaaacctataaaatctatagatatccaaagtccttttcaattattaaaacaaaaattaaattaaagcttgattttgaactaatttgtgattggcaaaaaggttgaaagttgttaaatcaggtcaaaattcacaaatagtcatttttcagttattgctatttctagagtcaatttttttaaaaaaaaaaatgggttaaaagcttcttacaaaacaagtatttgaaaactttagctcaaaacaaaatgtaacttacttatatttccttttttttatttttatattttctaataatattttcattgcagatggtcagttatagccggaagattacctggaagaacggataacgaaatcaagaactattggaatacacatattaaacgaaaactcattagtcgtggcattgatcctcaaactcaccgtccactcaacaacaacgccactaactcccacaccaccaccaatatcaccaccgcagtcacaaccaaaaacatcaacttggatttcacaaacgttgaccaaaaacaacccaatattatgattgccacgtcatcgtcatatgatgaaacaaaatgcaacagtggtacaactgaggaaacaaagccactagaaattattattccaaaaataccctcacaagttatgattaatcttgaactttcaattgggttgccgttacatactgatcatatttcttcaccagagtcaacggcctcatacaacttcttgaccaccgtagcaccgccgccaactgcggcggtaccggcggcggagatgatggcgaagactgtttgtttgtgttggcaaattggatatcatggtggtggcggtcagtggtgtggtaaatgcaaaaacacaaatggattttacagatattgctgacgtggcctctttttggatattattaagtaccttagtactaaatttatttttttccttaatattactttaccattttgtgataagtgaatattttgggttttgggttttaggtttagatatttttattttattttatttcatattttgcctcctaattgtaatcatctactcacaaaccttgtagtacatt

SEQ ID NO: 38 - DNA, 1111 bp, Solanum lycopersicum
ctcctcaaatccttgaatactcaaaggacaaaaataaaaaaaattcaaagtatgggacgttcaccttgttgtgaaaaagcacatacaaataaaggagcatggactaaagaagaagaccaacgcctcatcaattatatacgtgctcatggggaaggttgctggcgttctcttcctaaagctgcaggattgtcaagatgtggaaagagttgcagattgagatggataaattatttaaggcctgatctcaaaagagggaattttacagaagaagaagatgaattgataatcaaacttcatagtttgcttggaaacaaatggtcagttatagccggaagattacctggaagaacggataacgaaatcaagaactattggaatacacatattaaacgaaaactcattagtcgtggcattgatcctcaaactcaccgtccactcaacaacaacgccactaactcccacaccaccaccaatatcaccaccgcagtcacaaccaccaccaccacagccaaaaacatcaacttggatttcacaaacgttgaccaaaaacaacccaatattatgattgccacgtcatcgtcatatgatgaaacaaaatgcaacagtggtacaactgaggaaacaaagccactagaaattattattccaaaaataccctcacaagttatgattaatcttgaactttcaattgggttgccgttacatactgatcatatttcttcaccagagtcaacggcctcatacaacttcttgaccaccgtagcaccgccgccaactgcggcggtaccggcggcggagatgatggcgaagactgtttgtttgtgttggcaaatggatatcatggtggtggcggtcagtggtgtggtaaatgcaaaaacacaaatggattttacagatattgctgacgtggcctctttttggatattattaagtaccttagtactaaatttatttttttccttaatattactttaccattttgtgataagtgaatattttgggttttgggttttaggtttagatatttttattttattttatttcatattttgcctcctaattgtaatcatctactcacaaaccttgtagtacatttgttgcaacaacttac

SEQ ID NO: 39 - protein, 345 aa, Solanum lycopersicum
MGRSPCCEKAHTNKGAWTKEEDQRLINYIRAHGEGCWRSLPKAAGLSRCGKSCRLRWINYLRPDLKRGNFTEEEDELIIKLHSLLGNKWSVIAGRLPGRTDNEIKNYWNTHIKRKLISRGIDPQTHRPLNNNATNSHTTTNITTAVTTTTTTAKNINLDFTNVDQKQPNIMIATSSSYDETKCNSGTTEETKPLEIIIPKIPSQVMINLELSIGLPLHTDHISSPESTASYNFLTTVAPPPTAAVPAAEMMAKTVCLCWQMDIMVVAVSGVVNAKTQMDFTDIADVASFWILLSTLVLNLFFSLILLYHFVISEYFGFWVLGLDIFILFYFIFCLLIVIIYSQTL

SEQ ID NO: 40 - DNA, 19 bp, Artificial Sequence
  synthetic primer
gagtacatcgccgccaaca

SEQ ID NO: 41 - DNA, 23 bp, Artificial Sequence
  Synthetic primer
agtcacgttgcttgaatcatcct

SEQ ID NO: 42 - DNA, 21 bp, Artificial Sequence
  Synthetic primer
ccctggatggagctaaggaga

SEQ ID NO: 43 - DNA, 21 bp, Artificial Sequence
  Synthetic primer
ccttcacacccctcaacaaca

SEQ ID NO: 44 - DNA, 21 bp, Artificial Sequence
  synthetic primer
cagcctaaggaaggacttgca

SEQ ID NO: 45 - DNA, 24 bp, Artificial Sequence
  synthetic primer
gaaaatcgctgacaagacttcaga

SEQ ID NO: 46 - DNA, 21 bp, Artificial Sequence
  synthetic primer
tcacgtccacgtaacgttgtg

SEQ ID NO: 47 - DNA, 25 bp, Artificial Sequence
  Synthetic primer
tgatacgtctcatttttctccaatg

SEQ ID NO: 48 - DNA, 20 bp, Artificial Sequence
  Synthetic primer
cacactttggctcgcaaaca

SEQ ID NO: 49 - DNA, 22 bp, Artificial Sequence
  Synthetic primer
catatcccataggaggccgata

SEQ ID NO: 50 - protein, 20 aa, Artificial Sequence
  Synthetic primer
TTACCCTGGCGTTGAACACA

SEQ ID NO: 51 - DNA, 20 bp, Artificial Sequence
  Synthetic primer
tgctcatcgctccaatcatg

SEQ ID NO: 52 - DNA, 25 bp, Artificial Sequence
  Synthetic primer
tgacctacccaatgtcatcaaagat

SEQ ID NO: 53 - DNA, 23 bp, Artificial Sequence
  Synthetic primer
gaatgattagctccccttgagga

SEQ ID NO: 54 - DNA, 23 bp, Artificial Sequence
  Synthetic primer
aaccccactgctaaggctatttt

SEQ ID NO: 55 - DNA, 24 bp, Artificial Sequence
  Synthetic primer
gacaattacccccaaatgtcctaa

SEQ ID NO: 56 - DNA, 21 bp, Artificial Sequence
  Synthetic primer
tggtcaccgtggaggagtatc

SEQ ID NO: 57 - DNA, 21 bp, Artificial Sequence
  Synthetic primer
gatcgtagctggaccctctgc

SEQ ID NO: 58 - DNA, 23 bp, Artificial Sequence
  Synthetic primer
gcatatccaccattttttccggc

SEQ ID NO: 59 - DNA, 21 bp, Artificial Sequence
  Synthetic primer
cccacaatgtaagcccagccc

SEQ ID NO: 60 - DNA, 20 bp, Artificial Sequence
  Synthetic primer
cacggaacgaatggcattta

SEQ ID NO: 61 - DNA, 23 bp, Artificial Sequence
  Synthetic primer
ttccagatgcatgcaagtagaga

SEQ ID NO: 62 - DNA, 20 bp, Artificial Sequence
  Synthetic primer
ttggcgatcccttcaagaaa

SEQ ID NO: 63 - DNA, 20 bp, Artificial Sequence
  Synthetic primer
agagctgtcacggccttctc

SEQ ID NO: 64 - DNA, 24 bp, Artificial Sequence
  Synthetic primer
cccttgttccacctaagtaccatt

SEQ ID NO: 65 - DNA, 21 bp, Artificial Sequence
  Synthetic primer
gtgcttctgggagtgcaaaga

SEQ ID NO: 66 - DNA, 20 bp, Artificial Sequence
  Synthetic primer
ctgttcagcccgttgaaggt

SEQ ID NO: 67 - DNA, 22 bp, Artificial Sequence
  Synthetic primer
accactgcttgatgatcagcat

SEQ ID NO: 68 - DNA, 20 bp, Artificial Sequence
  Synthetic primer
attcgccgacggtactaacg

SEQ ID NO: 69 - DNA, 19 bp, Artificial Sequence
  Synthetic primer
atcgccgatgttgaaaacg

SEQ ID NO: 70 - DNA, 20 bp, Artificial Sequence
  Synthetic primer
gagcatgaagttgggccaat

SEQ ID NO: 71 - DNA, 20 bp, Artificial Sequence
  Synthetic primer
tggtgggttggcctcattaa

SEQ ID NO: 72 - DNA, 20 bp, Artificial Sequence
  Synthetic primer
ttggtttggaaggccatgaa

SEQ ID NO: 73 - DNA, 21 bp, Artificial Sequence
  Synthetic primer
aaatcaggccttggacatggt

SEQ ID NO: 74 - DNA, 25 bp, Artificial Sequence
  Synthetic primer
tcacaagcctacttaatttgttcca

SEQ ID NO: 75 - DNA, 24 bp, Artificial Sequence
  Synthetic primer
gctcgagggaaagttctagatgaa

SEQ ID NO: 76 - DNA, 19 bp, Artificial Sequence
  Synthetic primer
tgggatggcgtcaaacaag

SEQ ID NO: 77 - DNA, 22 bp, Artificial Sequence
  Synthetic primer
ccctgtttcctcctctgcttct

SEQ ID NO: 78 - DNA, 25 bp, Artificial Sequence
  Synthetic primer
tgcaggattcagttcagtgatagag

SEQ ID NO: 79 - DNA, 25 bp, Artificial Sequence
  Synthetic primer
tcatatccccactcactagttttgc

SEQ ID NO: 80 - DNA, 22 bp, Artificial Sequence
  Synthetic primer
gcaatcttctggtcaactgcag

SEQ ID NO: 81 - DNA, 19 bp, Artificial Sequence
  Synthetic primer
cccgcctgaacagtcttcc

SEQ ID NO: 82 - DNA, 26 bp, Artificial Sequence
  Synthetic primer
gaatatatgccaagttgtagcaagtc

SEQ ID NO: 83 - DNA, 25 bp, Artificial Sequence
  Synthetic primer
cacaaaaaagtgatgatcatgaaag

SEQ ID NO: 84 - DNA, 21 bp, Artificial Sequence
  Synthetic primer
ccaaacgaggacgcagtagaa

SEQ ID NO: 85 - DNA, 24 bp, Artificial Sequence
  Synthetic primer
atgccataacatctggtcatcaat

SEQ ID NO: 86 - DNA, 21 bp, Artificial Sequence
  Synthetic primer
gccagcttgtgatagtgccat

SEQ ID NO: 87 - DNA, 20 bp, Artificial Sequence
  Synthetic primer
agggcttcccttggcttcta

SEQ ID NO: 88 - DNA, 21 bp, Artificial Sequence
  Synthetic primer
atgggaagaacaccttgttgt

SEQ ID NO: 89 - DNA, 24 bp, Artificial Sequence
  Synthetic primer
tcaaaagcaatatataatgtcata

SEQ ID NO: 90 - DNA, 20 bp, Artificial Sequence
  Synthetic primer
agggcttcccttggcttcta

SEQ ID NO: 91 - DNA, 23 bp, Artificial Sequence
  Synthetic primer
attgatgaggcgttggtcttctt

SEQ ID NO: 92 - DNA, 20 bp, Artificial Sequence
  Synthetic primer
caaagtatgggacgttcacc

SEQ ID NO: 93 - DNA, 20 bp, Artificial Sequence
  Synthetic primer
ccaccatgatatccatttgc

SEQ ID NO: 94 - DNA, 25 bp, Artificial Sequence
  Synthetic primer
gtaaagattgcagttgtggaagtga

SEQ ID NO: 95 - DNA, 23 bp, Artificial Sequence
  Synthetic primer
ttcaagcccaaaaagtcataacc

SEQ ID NO: 96 - DNA, 22 bp, Artificial Sequence
  Synthetic primer
ccattatccttctctcaattgg

SEQ ID NO: 97 - DNA, 25 bp, Artificial Sequence
  Synthetic primer
ctatattgcaaagtttacaaccatg

SEQ ID NO: 98 - DNA, 20 bp, Artificial Sequence
  Synthetic primer
ccaggaaggacagcaaacga

SEQ ID NO: 99 - DNA, 23 bp, Artificial Sequence
  Synthetic primer
cgaggacgagaatgaggatgtag

SEQ ID NO: 100 - DNA, 21 bp, Artificial Sequence
  Synthetic primer
gaatactcctatgtgtgcatc

SEQ ID NO: 101 - DNA, 26 bp, Artificial Sequence
  Synthetic primer
caaaaataaaaattctttaattaagt

SEQ ID NO: 102 - DNA, 24 bp, Artificial Sequence
  Synthetic primer
tcctgatctcaaacatgggaaaat

SEQ ID NO: 103 - DNA, 20 bp, Artificial Sequence
  Synthetic primer
tttttcgggccattcttgac

SEQ ID NO: 104 - DNA, 24 bp, Artificial Sequence
  Synthetic primer
gatggtgcaagaagaaataatgag

SEQ ID NO: 105 - DNA, 24 bp, Artificial Sequence
  Synthetic primer
cgatagcgaaaatatgtcacattg

SEQ ID NO: 106 - DNA, 20 bp, Artificial Sequence
  Synthetic primer
tggctgttggagctctgtcc

SEQ ID NO: 107 - DNA, 23 bp, Artificial Sequence
  Synthetic primer
ctcttttcaaatcaggcctcaag

SEQ ID NO: 108 - DNA, 19 bp, Artificial Sequence
  Synthetic primer
ggccgggagaggcttttat

SEQ ID NO: 109 - DNA, 23 bp, Artificial Sequence
  Synthetic primer
ctatcatataccatccacaaaag

SEQ ID NO: 110 - DNA, 936 bp, Solanum lycopersicum
gagctaaataatttgaatcaatgggaagatcaccgtgttgtgaaaaagcacatacaaataaaggagcttggactaaagaagaagatgaacgacttatttcttatattaaaactcacggcgaaggttgctggagatcccttcctaaagctgccggacttctccgatgcggtaaaagttgccgtctccgatggattaattacttgagaccggaccttaaacgcggtaattttactgaagaagaagatgaactcattatcaaactccatagcctccttggtaacaaatggtcacttatagccggaagattaccaggaasaacagataatgagataaaaaattactggaatacgcacataagaaggaagcttttgagtcggggcattgatccaacgacacacaggcctgttaacgagcctggtacaacgcaaaaagtcacaacaatttcatttgcaggtggagatcataaaactaaagatattgaagaagatcataataagatgataaatgtcaaagctgaatctgggttgagtcaattagaagatgaaattattagtagcagtccatttcgagaacagtgtcctgatttaaatcttgagctcagaattagccctccttctctacaaaattaccaacatagcccctcaaggtgttttgcatgcagtttgggtatacaaaatagtaaagattgcaattgcagtaaaaataatattgcaagttataactttttaggattaaagagtaatggtgttttggactatagaactttagaaactaagtgaatttttattataaatctttttttccctcgtgtatttgggttaaaaaaacaagaagagagaatcgagaaagatattcctattagtttaagttctttcgaattttctcttatttgtaaaatttcaagtattactatatacgatatattatattaagttg

SEQ ID NO: 111 - protein, 252 aa, Solanum lycopersicum
MGRSPCCEKAHTNKGAWTKEEDERLISYIKTHGEGCWRSLPKAAGLLRCGKSCRLRWINYLRPDLKRGNFTEEEDELIIKLHSLLGNKWSLIAGRLPGRTDNEIKNYWNTHIRRKLLSRGIDPTTHRPVNEPGTTQKVTTISFAGGDHKTKDIEEDHNKMINVKAESGLSQLEDEIISSSPFREQCPDLNLELRISPPSLQNYQHSPSRCFACSLGIQNSKDCNCSKNNIASYNFLGLKSNGVLDYRTLETK

(19) United States
(12) Patent Application Publication
(10) Pub. No.: US 2011/0093986 A1
(43) Pub. Date: Apr. 21, 2011

(54) TOMATO SIMYB12 TRANSCRIPTION FACTOR AND GENETIC SELECTION THEREOF

(75) Inventors: Asaph Aharoni, Rehovot (IL); Avital Adato, Rehovot (IL); Tali Mandel, Rehovot (IL)

(73) Assignee: Yeda Research and Development Co., Ltd At The Weizmann Institute of Science, Rehovot (IL)

(21) Appl. No.: 12/999,814

(22) PCT Filed: Jun. 28, 2009
(86) PCT No.: PCT/IL2009/000642

§ 371 Date: Dec. 17, 2010

Related U.S. Application Data
(60) Provisional application No. 61/075,789, filed on Jun. 26, 2008.

(51) Int. Cl. A01H 5/00; C07H 21/04; C07H 21/00; C12Q 1/68

(52) U.S. Cl. 800/295; 536/23.6; 536/24.33; 435/6

(57) ABSTRACT

The present invention discloses that down regulation of the SlMYB12 transcription factor results in the colorless peel y phenotype in tomato fruit. The present invention provides polynucleotides encoding the tomato SlMYB12 transcription factor and genetic markers derived therefrom, useful in the breeding of tomato plants having the colorless peel phenotype and in the production of transgenic plants having altered flavonoid content.

### FIELD OF THE INVENTION

The present invention relates to a tomato SlMYB12 transcription factor associated with the tomato colorless peel phenotype and to genetic markers derived therefrom, useful in the breeding of tomato plants having the colorless peel phenotype and in the production of transgenic plants having altered flavonoid content.

### BACKGROUND OF THE INVENTION

Most aerial plant surfaces are covered with a cuticle, a heterogeneous layer composed mainly of lipids, namely cutin and waxes. The cuticle is a unique surface structure that plays an important role in organ development and the protection against biotic and abiotic stress conditions. Cutin is the major component of the cuticle; embedded in the cutin matrix are cuticular waxes, which are complex mixtures of very long chain fatty acid derivatives. In many species these also include triterpenoids and other secondary metabolites, such as sterols, alkaloids and flavonoids.

The yellow flavonoid naringenin chalcone (NarCh) accumulates to almost 1% of the dry cuticlar weight in tomato fruit. NarCh is the first intermediate in the biosynthesis of flavonols. It is produced by chalcone synthase (CHS) from p-coumaroyl-CoA and malonyl-CoA and subsequently converted into naringenin (Nar) by chalcone isomerase (CHI) (Muir S. R. et al., 2001. Nat. Biotechnol. 19: 470-474). Apart from NarCh, various other flavonoids accumulate in tomato fruit. The flavonol rutin (quercetin-3-rutinoside), and to a lesser extent kaempferol-3-O-rutinoside and quercetin trisaccharide, are predominantly produced in the tomato peel, while the fruit flesh tissues accumulate only minute amounts of flavonoids. These biochemical data correlate with the expression of the flavonoid biosynthesis genes in tomato fruit tissues, as only low levels of flavonoid-related transcripts were detected in the flesh (Bovy A. et al., 2002. Plant Cell 14: 2509-2526; Mintz-Oron S. et al., 2008. Plant Physiol. 147: 823-851). In the peel, significant levels of the transcripts encoding chalcone synthase (CHS), flavonone 3′ hydroxylase (F3′H), and flavonol synthase (FLS) enzymes could be detected, while chalcone isomerase (CHI) mRNA levels were barely detectable (Bovy et al., 2002, ibid). Low CHI expression might explain the accumulation of its substrate, NarCh, in the fruit peel. In fact, transgenic tomato plants expressing the petunia CHI gene displayed increased levels of fruit peel flavonols, mainly due to the accumulation of rutin, and a concomitant reduction of NarCh (Muir et al., 2001, ibid). Thus, unlike other steps in the flavonoid pathway, only the CHI reaction seems to be blocked in tomato fruit peel, whereas most of the pathway appears to be suppressed in the fruit flesh.

Various accumulation patterns during fruit development could be defined for different flavonoids. While flavonoids such as naringenin (Nar) and NarCh-hexose increased during fruit development, the levels of quercetin-trisaccharide decreased. Slimestad et al. (Slimestad, R. et al., 2008. J. Agric. Food Chem. 56: 2436-2441) determined the qualitative and quantitative flavonoid compositions of various tomato cultivars. Extensive characterization revealed that the total flavonoid content of different tomato types varied from about 4 to 26 mg per 100 g fruit weight, with NarCh being the predominant compound contributing 35-71% of the total flavonoid content. Iijima et al. (Iijima, Y. et al., 2008. Plant J. 54: 949-962) showed that the number of flavonoid increases during ripening and flavonoids are more abundant in peel tissues than in the flesh. Using a combined transcripts and metabolite analyses, Mintz-Oron et al. (2008, ibid) further demonstrated that the increase in activity of pathways generating cuticular lipids in tomato fruit peel precedes that of phenylpropanoid and flavonoid biosynthesis pathway.

Reducing the NarCh content in tomato fruit by CHI-over expression resulted in pink fruit with dull appearance (Verhoeyen M. E et al., 2002. J. Exp. Bot. 53: 2099-106). A similar pink phenotype was obtained upon RNAi-mediated down regulation of CHS, encoding the enzyme generating NarCh (Schijlen E. G. et al. 2007 Plant Physiol. 144: 1520-1530). Total flavonoid levels, transcript levels of both CHS1 and CHS2, as well as CHS enzyme activity were all significantly reduced in these latter transgenic tomato fruits. The highest RNAi-expressing lines produced extremely small and parthenocarpic fruits, and pollen tube growth was inhibited. SEM analysis revealed that epidermal cell development was strongly disturbed in the fruit of CHS RNAi plants, as the typical conical cells of tomato fruit epidermis were misshaped and collapsed.

The tomato y mutant was originally described in 1925 as carrying a monogenic recessive mutation leading to the formation of a colorless fruit peel, and was named “y” after the recessive colorless allele, in contrast to the dominant yellow “Y” allele (Lindstrom E. W. 1925. Inheritance in tomatoes (Genetics), pp. 305-317). In 1956 Rick and Butler (Rick C. M. and Butler, L. 1956. Adv. Genet. 8: 267-382) mapped the y mutation by linkage analysis to the cytogenetic band 30 on the S arm of chromosome 1, the 1-30 locus. The y-type fruit appearance is visible in numerous wild tomato species and cultivated varieties and is popular among commercial cultivars consumed in Asian countries.

The transcriptional regulation of the flavonoid biosynthesis pathway involves spatially and temporally coordinated expression of several transcription factors (Koes R. et al., 2005. Trends Plant Sci. 10: 236-242; Ramsay N. A. and Glover B. J. 2005. Trends Plant Sci. 10: 63-70; Lepiniec L. et al., 2006. Annu. Rev. Plant Biol. 57: 405-430). Studies in several plant species (e.g. maize, petunia, antirrhinum, Arabidopsis, tobacco, grape and apple) revealed that members of the R2R3-MYB gene family are required for the production of anthocyanins, proanthocyanidins and flavonols. Two of the best-studied examples of flavonoid-related transcription factors are the maize MYB-type C1 and the MYC-type LC genes. When specifically expressed in the fruit of transgenic tomato, both genes were shown to be required and sufficient for up-regulation of the flavonoid pathway in fruit flesh, which normally produces only low levels of flavonoids. A recent study showed that transgenic tomato lines over-expressing the Arabidopsis R2R3-MYB transcription factor PAP1, known to regulate the transcription of flavonoid pathway genes, accumulate increased levels of various flavonoid derivates (Iijima et al., 2008, ibid). Luo et al. (Luo J. et al., 2008. Plant J. 56: 316-326), published after the priority date of the present application studied the expression of AtMYB12, originally identified as a flavonol-specific transcriptional activator in Arabidopsis in tobacco and tomato. They showed that in tobacco, AtMYB12 is able to induce the expression of target genes, leading to the accumulation of very high flavonols levels, while in tomato AtMYB12 activated also the caffeoyl quinic acid biosynthetic pathway. These data confirmed previous observations that transcription factors may have different specificities for target genes in dissimilar plant species (Luo et al. 2008, ibid).

In tomato, T-DNA activation-tagging experiments identified a tomato MYB-type transcriptional regulator of anthocyanin biosynthesis, named Anthocyanin1 (ANT1), which shares high homology with the petunia AN2 protein regulating late anthocyanin pathway genes (Quattrocchio F. et al., 1999. Plant Cell 11: 1433-1444). Fruit of the ant1 mutant exhibited purple spotting on their epidermis. In an earlier study, Lin et al. (Lin Q. et al., 1996. Plant Mol. Biol. 30: 1009-1020) characterized the expression of 14 putative tomato MYB-type transcription factors, which showed a wide range of expression patterns including some transcripts with marked tissue specificity.

The fresh food market presents an ongoing demand for fruit and vegetables having elevated nutritional value. Flavonoids are an integral part of the human diet and there is increasing evidence that dietary polyphenols are likely candidates for the observed beneficial effects of a diet rich in fruit and vegetables in the prevention of several chronic diseases. Since tomato fruit is the main source for flavonoids consumption in the human diet there is considerable interest in enhancing the level of these bioactive molecules in this specie. Fresh food should also answer certain appearance quality, and consumers are looking for new and exotic varieties. For instance, the majority of commercial cultivars in the far-east have a pinkish appearance, which is based on the y genetic background. To answer the nutrition and appearance demands, there is a continuous attempt to elucidate the regulation and function of the flavonoid biosynthesis pathway.

Various strategies have been employed to elevate the flavonoid levels in plants. For example, U.S. Pat. No. 6,608,246 discloses a method for manipulating the production of flavonoids in tomatoes by expressing genes encoding chalcone isomerase. Tomato plants having altered flavonoid levels are also disclosed.

U.S. Pat. No. 7,208,659 discloses a method for manipulating the production of flavonoids in tomatoes taking the same approach, wherein the activity of chalcone synthase and flavonol synthase is increased.

U.S. Pat. No. 7,034,203 discloses a method for manipulating the production of flavonoids (other than anthocyanins) in plants by manipulating gene activity in the flavonoid biosynthetic pathway through the expression of two or more genes encoding transcription factors for flavonoid biosynthesis, particularly the maize transcription factors LC and C1. Also disclosed in this patent are transgenic tomato plants transformed with a combination of two or more maize transcription factors having altered flavonoid levels, particularly elevated levels of flavonols.

U.S. Patent Application Publication No. 20080134356 discloses a method for increasing at least one antioxidant level in a plant or plant product by expressing a polynucleotide that encodes a transcription factor, which is active in a flavonoid pathway. Particularly, the application discloses that over-expression of a novel and newly-identified gene, the mCai gene, in a plant, results in increased accumulation of chlorogenic acid and other related phenolics, which, in turn, increases the levels of beneficial antioxidant in the plant.

U.S. Patent Application Publication No. 20090100545 discloses the identification of a transcriptional regulon of 69 genes, which are involved in the synthesis of flavonoids, more particularly anthocyanins. These genes can be used to modulate the levels flavonoids in plants and plant cells.

Thus, various genes and transcription factors take part in the biosynthesis and regulation of the phenylpropanoid/flavonoid pathway. Different regulators control the expression of different branches of the pathway, and the regulators are specific for each plant species, tissue and developmental stage. Thus, there is a need for, and would be highly advantageous to have new genetic information useful in the production of tomato plants having fruit with a desired appearance and nutritional value.

### SUMMARY OF THE INVENTION

The present invention provides genetic markers for the production of plants having a desired phenotype and nutritional value using genetic selection techniques. Particularly, the present invention provides compositions and methods for detecting colorless peel y phenotype in tomato fruit, and transcriptomic and metabolic characterization of this phenotype. The present invention also provides methods of screening for genetic markers associated with the tomato y phenotype, genetic markers so revealed and methods of use thereof, particularly for detecting the colorless peel y phenotype in various plant organ and developmental stages. The present invention further provides isolated polynucleotides and transgenic plants comprising same having altered flavonoid content.

The present invention is based in part on the unexpected discovery that down regulation of a particular R2-R3 MYB transcription factor, SlMYB12, results in the y mutant phenotype in tomato fruit. Furthermore, the present invention now discloses a SlMYB12 allele (y-1) co-segregating with the y colorless peel phenotype that can be used as a marker for the phenotype. The y-1 allele comprises multiple sequence changes, most of them located within the first and the second introns. These sequence changes introduce premature polyadenylation sites within the coding sequence of exon 3.

Thus, according to one aspect, the present invention provides an isolated polynucleotide encoding a SlMYB12 variant transcript the polynucleotide comprising at least one alteration compared to the wild type SlMYB12, the wild type having the nucleic acid sequence set forth in SEQ ID NO:1, wherein the variant transcript results in down regulated expression and/or activity of the encoded SlMYB12 protein.

According to certain embodiments, the polynucleotide comprises at least one alteration in the promoter region, intron 1, intron 2, exon 3 or combinations thereof compared to the wild type SlMYB12 having the nucleic acid sequence set forth in SEQ ID NO:1.

According to other embodiments, the polynucleotide encoding the SlMYB12 variant transcript comprises a nucleic acid sequence as set forth in any one of SEQ ID NOs: 2-28.

According to certain embodiments, the alterations are present within intron 1 located between positions 905-994 of SEQ ID NO:1. According to a particular embodiment the isolated polynucleotide has a nucleic acid sequence as set forth in SEQ ID NO:29. According to other embodiments, the alterations are present in intron 2 located between positions 1474-1728 of SEQ ID NO:1. According to a particular embodiment the isolated polynucleotide has a nucleic acid sequence as set forth SEQ ID NO:30.

According to yet additional embodiments, the isolated polynucleotide is a SlMYB12 y-1 allele having a nucleic acid sequence as set forth in SEQ ID NO:31.

According to another aspect, the present invention provides a detecting agent capable of detecting a polynucleotide encoding a variant SlMYB12 transcript, the polynucleotide comprising at least one alteration compared to the wild type SlMYB12, the wild type having the nucleic acid sequence set forth in SEQ ID NO:1, wherein the variant transcript results in down regulated expression and/or activity of the encoded SlMYB12 protein.

According to certain embodiments, the polynucleotide comprises at least one alteration in intron 1, intron 2, exon 3 and combinations thereof compared to the wild type SlMYB12 having the nucleic acid sequence set forth in SEQ ID NO:1.

According to other embodiments, the detecting agent is a polynucleotide probe differentially hybridizable to the polynucleotide encoding the SlMYB12 variant compared to a wild-type SlMYB12 having the nucleic acid sequence as set forth in SEQ ID NO:1.

According to particular embodiments, the detecting agent is a polynucleotide probe differentially hybridizable to a polynucleotide having a nucleic acid sequence as set forth in any one of SEQ ID NOs:2-31 or part thereof compared to a wild-type SlMYB12 having the nucleic acid sequence as set forth in SEQ ID NO:1.

According to other embodiments, the detecting agent is a primer pair capable of selectively amplifying the polynucleotide encoding the SlMYB12 variant compared to a wild-type SlMYB12 having the nucleic acid sequence as set forth in SEQ ID NO:1.

According to particular embodiments, the detecting agent is a primer pair capable of selectively amplifying SlMYB12 variant having a nucleic acid sequence as set forth in any one of SEQ ID NOs: 2-31 or part thereof compared to a wild type SlMYB12 having the nucleic acid sequence as set forth in SEQ ID NO:1.

According to another aspect the present invention provides a method of screening for genetic markers indicative of the y mutant phenotype in a tomato plant, comprising (a) comparing the genomic polynucleotide sequence of the SlMYB12 gene having SEQ ID NO:1 of the wild type tomato plant or a fragment thereof to the genomic polynucleotide sequence of a SlMYB12 gene in a tissue sample obtained from a y phenotype tomato plant; (b) identifying alterations in the SlMYB12 genomic sequence of the y phenotype, wherein the alterations predict modification in the gene transcription and/or translation; wherein said alterations are genetic markers indicative of the y mutant phenotype.

According to certain embodiments, the alterations results in down regulation of the expression or activity of SlMYB 12 in the y phenotype tomato plant.

According to certain embodiments, the alteration is identified within a non-coding region selected from the group consisting of an intron and an upstream promoter region. According to certain currently preferred embodiments the alteration is identified in the promoter sequence.

Identification of the alteration in the SlMYB12 gene of the tomato y phenotype compared to the wild type gene can be performed by any method as is known to a person skilled in the art. According to certain embodiments, the alteration in the SlMYB12 sequence is determined by an assay selected from the group consisting of (a) observing shifts in electrophoretic mobility of single-stranded DNA on non-denaturing polyacrylamide gels; (b) hybridizing a SlMYB12 gene probe to genomic DNA isolated from the y phenotype tomato; (c) hybridizing an allele-specific probe to genomic DNA of said y phenotype tomato; (d) amplifying all or part of the SlMYB12 gene from said y phenotype tomato to produce an amplified sequence and sequencing the amplified sequence; (e) amplifying all or part of the SlMYB12 gene from said y phenotype tomato using primers for a specific SlMYB12 mutant allele; (f) molecularly cloning all or part of the SlMYB12 gene from said y phenotype tomato to produce a cloned sequence and sequencing the cloned sequence; (g) identifying a mismatch between (1) a SlMYB12 gene or a SlMYB12 mRNA isolated from said y phenotype tomato, and (2) a nucleic acid probe complementary to the tomato wild-type SlMYB12 gene sequence, when molecules (1) and (2) are hybridized to each other to form a duplex, (h) amplification of SlMYB12 gene sequences in said y phenotype tomato and hybridization of the amplified sequences to nucleic acid probes which comprise tomato wild-type SlMYB12 gene sequences, (i) amplification of SlMYB12 gene sequences in said tissue sample and hybridization of the amplified sequences to nucleic acid probes which comprise mutant SlMYB12 gene sequences, (j) screening for a deletion mutation in said y phenotype tomato, (k) screening for a point mutation in said y phenotype tomato, (l) screening for an insertion mutation in said y phenotype tomato, (m) in situ hybridization of the SlMYB12 gene of said y phenotype tomato with nucleic acid probes which comprise the SlMYB12 gene.

It is to be understood that any sequence alteration revealed by the methods of the present invention, a polynucleotide comprising same and a detecting agent capable of identifying such alteration are also encompassed within the scope of the present invention.

According to an additional aspect, the present invention provides a method for identifying a tomato plant capable of producing fruit having the colorless peel y phenotype comprising (a) providing a sample comprising genetic material from the plant before fruit are produced; (b) determining, in the sample, the sequence of the SlMYB12 gene or its transcript or a part thereof (c) comparing the sequence to the sequence of a tomato wild type SlMYB12 gene or transcript; and (d) detecting at least one alteration in the SlMYB12 sequence from said sample, wherein the alteration is indicative of the capability of the plant to produce fruit having a y phenotype.

According to one embodiment, the wild type SlMYB12 gene comprises a nucleic acid sequence as set forth in SEQ ID NO:1. According to other embodiments, the wild type SlMYB12 transcript comprises a nucleic acid sequence as set forth in SEQ ID NO:32.

According to certain embodiments, the at least one sequences alteration results in down regulation of the expression or activity of SlMYB12.

According to other embodiments, the alteration in SEQ ID NO:1 are present within the promoter region, intron 1, intron 2, exon 3 or combinations thereof compared to the wild type SlMYB12 having the nucleic acid sequence set forth in SEQ ID NO:1, said alterations predict modification in the gene transcription and/or translation.

According to particular embodiments, the SlMYB12 gene of the sample comprises a nucleic acid sequence as set forth in any one of SEQ ID NOs:2-31.

According to certain embodiments, identifying the at least one alteration is performed by a technique selected from the group consisting of, but not limited to, terminator sequencing, restriction digestion, allele-specific polymerase reaction, single-stranded conformational polymorphism analysis, genetic bit analysis, temperature gradient gel electrophoresis, ligase chain reaction and ligase/polymerase genetic bit analysis.

According to other embodiments, the alteration in the SlMYB12 sequence is identified by employing nucleotides with a detectable characteristic selected from the group consisting of inherent mass, electric charge, electric spin, mass tag, radioactive isotope type bioluminescent molecule, chemiluminescent molecule, tagged nucleic acid molecule, hapten molecule, protein molecule, light scattering/phase shifting molecule and fluorescent molecule.

According to a further aspect the present invention provides an isolated polynucleotide encoding a wild type SlMYB12 polypeptide having SEQ ID NO:33.

According to the certain embodiments, the polynucleotide comprises a nucleic acid sequence as set forth in SEQ ID NO:1. According to other embodiments, the polynucleotide comprises a nucleic acid sequence as set forth in SEQ ID NO:32. According to other embodiments, the polynucleotide consists of the nucleic acid sequence set forth in any one of SEQ ID NO:1 and SEQ ID NO:32. DNA constructs and/or expression vectors comprising same are also encompassed within the scope of the present invention.

According to yet further aspect the present invention provides a transgenic plant comprising at least one cell comprising in its genome an exogenous polynucleotide encoding SlMYB12, wherein the plant has an elevated content of at least one phenylpropanoid selected from the group consisting of flavonoids, chlorogenic acid and derivative thereof compared to a non-transgenic plant.

According to certain embodiments, the transgenic plant contains a polynucleotide encoding SlMYB12 comprising a nucleic acid sequence as set forth in SEQ ID NO:1. According to other embodiments, the polynucleotide encoding SlMYB12 comprises a nucleic acid sequence as set forth in SEQ ID NO:32.

According to other embodiments, the flavonoids are flavonones. According to other embodiments, the flavonoids are selected from the group consisting of naringenin, naringenin chalcone, eridictyol, phloretin, resveratrol, Quercetin-hexose-deoxyhexose-pentose (Q-triscch) and Quercetin rutinoside (Rutin).

According to certain embodiments, the polynucleotide encoding SlMYB12 is incorporated into a DNA construct enabling its expression in the plant cell. According to one embodiment, the DNA construct comprises at least one expression regulating element selected from the group consisting of a promoter, an enhancer, an origin of replication, a transcription termination sequence, a polyadenylation signal and the like.

According to some embodiments, the DNA construct comprises a promoter. The promoter can be constitutive, induced or tissue specific promoter as is known in the art. According to certain embodiments, the promoter is a constitutive promoter operable in a plant cell. According to typical embodiments, the promoter is fruit specific promoter. According to another embodiment, the DNA construct further comprises transcription termination and polyadenylation sequence signals.

According to certain embodiment, the plant is of the Solanaceae family. According to typical embodiments, the plant is a tomato (Solanum lycopersicum) plant.

Optionally, the DNA construct further comprises a nucleic acid sequence encoding a detection marker enabling convenient detection of the recombinant polypeptides expressed by the plant cell. Any detection marker as in known in the art may be used according to the teachings of the present invention, including, but not limited to, markers comprising epitope tag, markers that confer resistance to an antibiotic, markers that confer resistance to a herbicide and the like.

The polynucleotides of the present invention and/or the DNA constructs comprising same can be incorporated into a plant transformation vector.

Other objects, features and advantages of the present invention will become clear from the following description and drawings.

### BRIEF DESCRIPTION OF THE FIGURES

FIG. 1 shows Real Time RT-PCR expression analyses of the SlTHM27 (FIG. 1A) and SlMYB4-like (FIG. 1B), the tomato homologues of Arabidopsis AtMYB4. Expression of the SlMYB12 paralogue (SlMYB12-like) is presented in FIG. 1C.

FIG. 2 demonstrates that the transgenic amiR-SlMYB12 lines exhibit y-like phenotype. FIG. 2A: Schematic description of the SlMYB12 gene, with the black box indicating the location of the amiR-SlMYB12 target sequence along the SlMYB12 gene. Arrows indicate the position of RT-PCR primers, and the sequence alignment in the right demonstrates the specificity of this artificial micro-RNA. FIG. 2B: Expression of the amiR-SlMYB12 precursor in samples extracted from leaves of 35S:amiR-SlMYB12 transgenic lines and non-transgenic controls. FIG. 2C: Fruit of 35S:amiRSlMYB12 transgenic lines display colorless-peel. FIG. 2D: RT-PCR relative expression analysis of phenylpropanoid/flavonoid-related regulators and structural genes in fruit peel of wt and 35S:amiRSlMYB12 transgenic line. Indicated by asterisks are significantly reduced levels analyzed by student's t-test (n=3; P<0.05; bars indicate standard errors). FIG. 2E: Principal Component Analysis (PCA) of metabolic profiles obtained by UPLC-QTOF-MS analysis, with peel samples of wt cv. Ailsa Craig (AC) and cv. MicroTom (MT), y mutant and an amiRSlMYB12 transgenic line at red stage of fruit development. Analysis was performed with TMEV program using normalized and log transformed data. FIG. 2F: Total Ion Chromatograms (TIC) of wt (cv. MT) and 35S:amiR-SlMYB12 peels at the red stage of fruit development, acquired in the negative mode using UPLC-QTOF-MS (in relative intensity, 100% corresponds to 6.14×104 counts). FIG. 2G: Relative levels of NarCh in cv. MT and 35S:amiR-SlMYB12, expressed as chromatographic peak areas, calculated for m/z 271.06 Da (n=5).

FIG. 3 demonstrates Gene expression alterations in the y mutant fruit as revealed by array analysis. FIG. 3A: Functional categories distribution among wt and y mutant transcripts, differentially expressed at the three latest stages of fruit development. FIG. 3B: The expression profile (obtained by array analysis) of genes belonging to cluster 14 (total 38 members) in the peel tissue of the y mutant and wild type (wt) fruit. In the y mutant, array analysis was carried out on three out of the 5 stages of fruit development that were examined in the wt fruit.

FIG. 4 shows differences between metabolic profiles of wt and y mutant peel and flesh tissues detected by Principal Component Analysis (PCA) analyses of GC-MS and LC-QTOF-MS data sets. FIG. 4A: PCA of metabolic profiles obtained by GC-MS analysis, with samples of wt and y peel and flesh tissues along five stages of fruit development (n=3). FIG. 4B: PCA of metabolic profiles obtained by UPLC-QTOF-MS analysis, with samples of wt and y peel and flesh tissues along five stages of the fruit development (n=3). FIG. 4C: PCA of metabolic profiles obtained by UPLCQTOF-MS analysis, with samples of wt and y peel and flesh tissues along the latest, three stages of fruit development (n=3). Distinguished metabolic profiles that correspond to particular stages of fruit development in y and wt are encircled in FIGS. 4A, B and C.

FIG. 5 summarizes GC-MS analyses showing metabolites that their level was found to be significantly different between wt and y mutant peel and/or flesh, in at least one tested stage of fruit development. Indicated by asterisks are significant differences as analyzed by a 2 way Anova test and post-hoc analysis, see Materials and Methods (n=3 for each sample). The Y axes indicate relative quantification of the metabolites by the normalization of their response values to the Ribitol internal standard (IS).

FIG. 6 demonstrates Real Time-PCR relative expression analyses of selected transcripts from the phenylpropanoids pathway in wt and y mutant tomato peels at the breaker stage of fruit development. Indicated by asterisks are significant differences analyzed by student's t-test (n=3; P<0.05; bars indicate standard errors).

FIG. 7 demonstrates Real Time-PCR relative expression analyses of selected transcripts from the phenylpropanoids pathway in wt and y mutant tomato flesh tissues at the breaker stage of fruit development. Indicated by asterisks are significant differences analyzed by a student's t-test (n=3; P<0.05; bars indicate standard errors).

FIG. 8 shows phylogeny and expression analyses of putative phenylpropanoid/flavonoid-related transcription factors genes. FIG. 8A: Phylogenetic analysis of the putative tomato regulators reported herein and known phenylpropanoid/flavonoid related transcription factors from other species. The ClustalX and NJplot software were used to compute the tree and its significance (bootstrap) values. FIG. 8B-C: Real Time RT-PCR relative expression analyses of selected tomato transcription factors putatively related to the regulation of the phenylpropanoid/flavonoid pathway in wt and in y mutant peel (FIG. 8B) and flesh (FIG. 8C) tissues at the breaker stage of fruit development. Indicated by asterisks are significant differences analyzed by student's t-test (n=3; P<0.05; bars indicate standard errors). Gene identifiers and primers are listed in Table 4. FIG. 8D: Real Time RT-PCR relative expression analysis of SlMYB12 in wt fruit tissues along five developmental stages reveals a peel-associated expression pattern. IG—Immature Green; MG—Mature Green; Br, Breaker; Or, Orange; Re, Red, stages of fruit development.

FIG. 9 schematically shows the chromosomal location of SlMYB12. FIG. 9A: BstBI digestion of SlMYB12 genomic fragments amplified from the tomato set of interspecific introgression lines between cv. M82 and Lycopersicon pennellii. FIG. 9B: The Il1-1 pennellii chromosome segment, which does not overlap with other introgression lines resides between 17 cM to 41 cM.

FIG. 10 shows sectorial phenotype complementation in a transgenic y line constitutively expressing the SlMYB12 gene under the 35S CaMV promoter (35S:SlMYB12). UPLC-PDA analysis of red fruit peels revealed significantly different levels of flavanoids between regions of phenotype complementation in peels of the 35S:SlMYB12 line and those of the y mutant (n=3; P<0.01; bar represent standard error). The UPLC (Waters, Acquity) instrument used in this analysis is equipped with an Acquity 2996 PDA detector and the samples run in LC conditions as described for the UPLCQTOF-MS analysis. Compounds peak areas were determined by the Empower 2 software (Waters) at 370 nm for Naringenin Chalcone (NarCh) and at 256 nm for Quercetin-hexose-deoxyhexose-pentose (Q-triscch) and Quercetin rutinoside (Rutin).

FIG. 11 demonstrates that the y mutation affects metabolism and gene expression in different plant organs apart from fruit. Real-Time PCR expression analyses of selected phenylpropanoid/flavonoid-related transcripts (trans.) in: FIG. 11A: young leaves. FIG. 11B: fully expanded leaves. Indicated by asterisks are significant differences analyzed by student's t-test (n=3; P<0.05; bars indicate standard errors). Gene identifiers and primers are listed in Table 4. C, PCA of metabolic profiles obtained by UPLC-QTOF-MS analysis clearly distinguish between samples of wt and y mutant roots.

### DETAILED DESCRIPTION OF THE INVENTION

The present invention provides detailed analyses of the tomato colorless peel y mutant. The fruit produced by the y mutant exhibit a severe reduction in NarCh, the yellow flavonoid pigment that typically accumulates up to 1% of the cuticle mass. Detailed characterization of y fruit tissues as well as some other plant parts, revealed extensive alterations in transcripts and metabolites associated with the phenylpropanoid/flavonoid pathway, which were not restricted to the fruit peel. The present invention discloses for the first time the association of the transcription factor SlMYB12 with the y mutant phenotype of tomato plant, which produce fruit with colorless fruit peel having a pinkish appearance.

### Definitions

The term “plant” is used herein in its broadest sense. It includes, but is not limited to, any species of woody, herbaceous, perennial or annual plant. It also refers to a plurality of plant cells that are largely differentiated into a structure that is present at any stage of a plant's development. Such structures include, but are not limited to, a root, stern, shoot, leaf, flower, petal, fruit, etc. In particular embodiments, the term relates to plants of the Solanaceae family, particularly tomato (Solanum lycopersicum).

As used herein, the terms “colorless peel phenotype” “y mutant”, “y phenotype” and “colorless peel y phenotype/mutant” are used herein interchangeably, and relate to a tomato fruit or tomato plant capable of producing fruit having colorless peel compared to the yellow-colored normal peel, which result in the pinkish-pink appearance of the fruit.

The term phenylpropanoids refer to classes of plant-derived organic compounds that are biosynthesized from the amino acid phenylalanine. The phenylpropanoids have a wide variety of functions in the plant, including defense against herbivores, microbial attack, or other sources of injury; as structural components of cell walls; as protection from ultraviolet light; as pigments; and as signaling molecules.

As used herein, the term “gene” has its meaning as understood in the art. In general, a gene is taken to include gene regulatory sequences (e.g. promoters, enhancers, etc.) and/or intron sequences, in addition to coding sequences (open reading frames). It will further be appreciated that definitions of “gene” include references to nucleic acids that do not encode proteins but rather encode functional RNA molecules such as microRNAs (miRNAs), tRNAs, etc. The term “transcript” as used herein refers to a portion of the gene that encodes a protein.

The term “allele” as used herein refers to one of the different forms of a gene or DNA sequence that can exist at a single locus within the genome.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

As used interchangeably herein, the terms “oligonucleotides”, “polynucleotides” and “nucleic acid sequence” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modification, including, for example, analogous linking groups, purine, pyrimidines, and sugars. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

As used herein, the term “isolated” means 1) separated from at least some of the components with which it is usually associated in nature; 2) prepared or purified by a process that involves the hand of man; and/or 3) not occurring in nature. Particularly, the term is used herein to describe a polynucleotide of the invention which has been to some extent separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially isolated when at least about 50%, preferably 60 to 75% of a sample exhibits a single polynucleotide sequence and conformation (linear versus covalently closed). The degree of polynucleotide isolation or homogeneity may be indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art.

The term primer refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The term “probe” or “hybridization probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified by hybridization. “Probes” or “hybridization probes” are nucleic acids capable of binding in a base-specific manner to a complementary strand of nucleic acid. Such probes include peptide nucleic acids, as described in Nielsen et al. (1991. Science 254:1497-1500). Hybridizations are usually performed under “stringent conditions”, for example, at a salt concentration of no more than 1M and a temperature of at least 25° C. For example, conditions of 5×SSPE 750 mM NaCl, 50 mM NaPhosphate, 5 mM EDTA, pH 7.4) and a temperature of 25° C. to 30° C. are suitable for allele-specific probe hybridizations. Although this particular buffer composition is offered as an example, one skilled in the art could easily substitute other compositions of equal suitability.

The term “sequencing” as used herein means a process for determining the order of nucleotides in a nucleic acid. A variety of methods for sequencing nucleic acids are well known in the art. Such sequencing methods include the Sanger method of dideoxy-mediated chain termination as described, for example, in Sanger et al. 1977. Proc Natl Acad Sci 74:5463, which is incorporated herein by reference (see, also, “DNA Sequencing” in Sambrook et al. (eds.), Molecular Cloning: A Laboratory Manual (Second Edition), Plainview, N.Y.: Cold Spring Harbor Laboratory Press (1989), which is incorporated herein by reference). A variety of polymerases including the Klenow fragment of E. coli DNA polymerase I; Sequenase™ (T7 DNA polymerase); Taq DNA polymerase and Amplitaq can be used in enzymatic sequencing methods. Well known sequencing methods also include Maxam-Gilbert chemical degradation of DNA (Maxam and Gilbert, 1980. Methods Enzymol. 65:499), which is incorporated herein by reference; and “DNA Sequencing” in Sambrook et al., 1989. ibid). One skilled in the art recognizes that sequencing is now often performed with the aid of automated methods.

The term “construct” as used herein refers to an artificially assembled or isolated nucleic acid molecule which includes the gene of interest. In general a construct may include the gene or genes of interest, a marker gene which in some cases can also be the gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

The terms “promoter element,” “promoter,” or “promoter sequence” as used herein, refer to a DNA sequence that is located upstream to the 5′ end (i.e. proceeds) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”. New promoters of various types useful in plant cells are constantly being discovered; numerous examples may be found in Okamuro J K and Goldberg R B (1989) Biochemistry of Plants 15:1-82.

As used herein, the term an “enhancer” refers to a DNA sequence which can stimulate promoter activity, and may be an innate element of the promoter or a heterologous element inserted to enhance the level or tissue-specificity of a promoter.

The term “expression”, as used herein, refers to the production of a functional end-product e.g., an mRNA or a protein.

The term “transgenic” when used in reference to a plant or seed (i.e., a “transgenic plant” or a “transgenic seed”) refers to a plant or seed that contains at least one heterologous transcribable gene in one or more of its cells. The term “transgenic plant material” refers broadly to a plant, a plant structure, a plant tissue, a plant seed or a plant cell that contains at least one heterologous gene in at least one of its cells.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. Transient transformation may be detected by, for example, enzyme-linked immunosorbent assay (ELISA), which detects the presence of a polypeptide encoded by one or more of the exogenous polynucleotides. Alternatively, transient transformation may be detected by detecting the activity of a marker protein (e.g. β-glucuronidase) encoded by at least one of the exogenous polynucleotides.

The term “transient transformant” refers to a cell which has transiently incorporated one or more exogenous polynucleotides. In contrast, the term “stable transformation” or “stably transformed” refers to the introduction and integration of one or more exogenous polynucleotides into the genome of a cell. Stable transformation of a cell may be detected by Southern blot hybridization of genomic DNA of the cell with nucleic acid sequences which are capable of binding to one or more of the exogenous polynucleotides. Alternatively, stable transformation of a cell may also be detected by enzyme activity of an integrated gene in growing tissue or by the polymerase chain reaction of genomic DNA of the cell to amplify exogenous polynucleotide sequences. The term “stable transformant” refers to a cell which has stably integrated one or more exogenous polynucleotides into the genomic or organellar DNA. It is to be understood that a plant or a plant cell transformed with the nucleic acids, constructs and/or vectors of the present invention can be transiently as well as stably transformed.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

### PREFERRED MODES FOR CARRYING OUT THE INVENTION

Down-Regulation of SlMYB12 Underlies the y Mutant Phenotype

The extensive alterations revealed by transcriptome and metabolome analyses in the tomato y mutant implied the deficiency in a regulatory factor rather than a single, structural gene. Thus, the analyses of the present invention focused on tomato orthologs/homologs of particular members of the MYB and MYC (bHLH) family of transcription factors that were previously shown to be associated with the control of the phenylpropanoid and flavonoid pathways in other plant species.

The present invention now discloses that three of the transcription factors differentially expressed in the y mutant compared to the wild type (SlTHM27, SlMYB4-like and SlMYB12) are significantly down-regulated in the y mutant fruit peel. Of these factors, SlTHM27 has been previously isolated and its transcript was sequenced (SEQ ID NO:34), encoding a protein having SEQ ID NO:35. The present invention now discloses the genomic sequence of the tomato homologue, having a nucleic acid sequence as set forth in SEQ ID NO:36.

The full length sequence of the tomato SlMYB4-like and SlMYB12 transcription factors is disclosed herein for the first time. The genomic sequence of SlMYB4-like comprises a nucleic acid sequence as set forth in SEQ ID NO:37, and its transcript comprises the nucleic acid sequence set forth in SEQ ID NO:38. The encoded protein comprises the amino acid sequence set forth in SEQ ID NO:39. SlMYB12 genomic sequence comprises the nucleic acid sequence set forth in SEQ ID NO:1, its transcript comprises the nucleic acid sequence set forth in SEQ ID NO: 32, and the encoded protein comprises the amino acid sequence set forth in SEQ ID NO:33. Furthermore, the present invention now discloses additional tomato SlMYB4-like transcription factor (SEQ ID NO:110), encoding a protein having the amino acid sequence se forth in SEQ ID NO:111.

Only one of these factors, the peel-associated SlMYB12, maps to a genomic region on chromosome 1 previously reported to harbor the mutation underlying the y mutant phenotype (Rick and Butler 1956, ibid). Thus, the correlation of this transcription factor with the y-mutant phenotype was further investigated.

Artificial microRNA targeted to the wild type SlMYB12 (35S:amiR-SlMYB12) was shown to significantly down-regulate (at least 5 fold lower than that of the wt) the SlMYB12 transcript levels in the breaker stage peel of the 35S:amiR-SlMYB12 plants. No significant difference was detected in the levels of SlMYB12 closest paralogue, SlMYB12-like, confirming the specificity of the synthetic micro RNA construct. This down regulation resulted in induction of a y-mutant like phenotype: the transcript and metabolic alterations detected in the 35S:amiR-SlMYB12 fruit peel compared to a wild type were very similar to those detected in the y mutant. The differences that were found in the accumulation of peel flavonols in the y mutant and the 35S:amiR-SlMYB12 fruit peels may be attributed to the lower levels of total polyphenols known to be a feature common to larger-fruited tomato varieties, such as cv. AC, in comparison to cherry tomatoes, such as cv. MT (Raffo A. et al., 2002. J. Agric. Food Chem. 50: 6550-6556).

The linkage of the transcription factor SlMYB12 with the y phenotype was further demonstrated by the finding of an additional SlMYB12 allele (y-1) that co-segregated with the colorless-peel phenotype among a large unrelated introgression population (>100 lines). All these lines were found to carry the same combination of sequence changes in introns and exons of their MYB12 gene, defining the new allele. These changes, including nucleotide insertion, deletions and replacements in the SlMYB12 wilt type genomic sequence are summarized in Tables 1-3 hereinbelow. The positions of the alterations are counted from the ATG at the beginning of the coding region (A being position No. 1).

TABLE 1
Alterations in the wild type S1MYB12
gene (SEQ ID NO: 1) - insertions

| Variant | SEQ ID NO. |
|---|---|
| Extra C between position 187 and 188 | 2 |
| Extra C between position 199-200 | 3 |
| Extra A between position 944-945 | 4 |
| Extra T between position 1064-1065 | 5 |
| Insertion of TTA between positions 1115-1116 | 6 |

TABLE 2
Alterations in the wild type
S1MYB12 gene (SEQ ID NO: l)-deletions

| | Alteration | SEQ |
|---|---|---|
| | Compared | ID |
| Variant | to Wild type | NO. |
| Nucleotides | GAAAAATAAT | 7 |
| at positions | ATTCAAAATT | |
| 777-796 are | are deleted | |
| missing | | |
| C at position 873 | C | 8 |
| is missing | is deleted | |
| Nucleotides at | TTGTCAAAT | 9 |
| positions | ATGATTCTC | |
| 905-922 are | are deleted | |
| missing | | |
| Nucleotides at | TTGAA | 10 |
| positions | are deleted | |
| 1033-1037 are | | |
| missing | | |
| Nucleotides at | AAATTTTTAT | 11 |
| positions | are deleted | |
| 1270-1279 are | | |
| missing | | |
| Nucleotides at | TAA | 12 |
| positions | are deleted | |
| 1626-1628 are | | |
| missing | | |

TABLE 3
Alterations in the wild type S1MYB12 gene
(SEQ ID NO: 1) - nucleotide replacement

| Wild type Position | Wild type | y-mutant | Variant SEQ ID NO. |
|---|---|---|---|
| 177 | A | T | 13 |
| 189 | G | A | 14 |
| 845 | A | C | 15 |
| 852 | C | T | 16 |
| 960 | C | T | 17 |
| 961 | A | C | 18 |
| 1042 | A | G | 19 |
| 1048 | A | G | 20 |
| 1062 | C | T | 21 |
| 1364 | A | T | 22 |
| 1393 | A | G | 23 |
| 1394 | G | A | 24 |
| 1418 | T | C | 25 |
| 1419 | A | G | 26 |
| 1452 | T | G | 27 |
| 1563 | C | T | 28 |

RACE analysis of the SlMYB12 transcript in the y-1 mutant allele revealed a set of pre-maturely poly-adenylated transcripts, which were poly-adenylated in the coding region of exon three. These most probably occurred as a result of sequence changes that introduced one or several new polyadenylation signals. One of the alternatively adenylated variants seems to be similar to the short wt version. However, this transcript comprises only a small portion (3/20) of the total RACE transcripts set and the function of its putative protein product is likely to be effected from the T331A missense, which is expected to disturb the formation of a helix structure at the C-terminus of the protein.

Taken together, these data clearly show that the SlMYB12 transcription factor regulates the y-mutant phenotype in tomato.

As exemplified hereinbelow, alterations in the y mutant are manifested also in the root and fully expended leaves, in which SlMYB12 is also expressed. Furthermore, the peel-associated expression pattern of SlMYB12 is in accordance with the predominant accumulation of flavonoids in tomato fruit peel. The relative high expression levels of SlMYB12 at early stages of fruit development prior to flavonoids accumulation can account for early alterations in primary metabolites, such as organic and amino acids. Similar wide range effects on target genes involved in both primary and secondary metabolism was previously demonstrated for regulators of glucosinolate biosynthesis in Arabidopsis.

Thus, according to one aspect, the present invention provides an isolated polynucleotide encoding a SIMYB12 variant transcript, the polynucleotide comprising at least one alteration compared to the wild type SlMYB12, the wild type having the nucleic acid sequence set forth in SEQ ID NO:1, wherein the variant transcript results in down regulated expression and/or activity of the encoded SlMYB12 protein.

According to certain embodiments, the polynucleotide comprising at least one alteration in the promoter region intron 1, intron 2, exon 3 or combinations thereof compared to the wild type SlMYB12 having the nucleic acids sequence set forth in SEQ ID NO:1.

According to other embodiments, the polynucleotide encoding a SIMYB12 variant transcript or protein comprises at least one alteration selected from those listed in Tables 1-3. According to other embodiments, the polynucleotide encoding a SlMYB12 variant transcript or polypeptide comprises a nucleic acid sequence as set forth in any one of SEQ ID NOs: 2-28.

According to certain embodiments, the alterations are present within intron 1 located between position 905-994 of SEQ ID NO:1. According to a particular embodiment the isolated polynucleotide has a nucleic acid sequence as set forth in SEQ ID NO:29. According to other embodiments, the alterations are present in intron 2 located between positions 1474-1728 of SEQ ID NO:1. According to a particular embodiment the isolated polynucleotide has a nucleic acid sequence as set forth SEQ ID NO:30.

According to yet additional embodiments, the isolated polynucleotide is a SlMYB12 y-1 allele having a nucleic acid sequence as set forth in SEQ ID NO:31.

According to another aspect, the present invention provides a detecting agent capable of detecting a polynucleotide encoding a variant SlMYB12 transcript, the polynucleotide comprising at least one alteration compared to the wild type SlMYB12, the wild type having the nucleic acid sequence set forth in SEQ ID NO:1, wherein the variant transcript results in down regulated expression and/or activity of the encoded SlMYB12 protein.

According to certain embodiments, the polynucleotide comprising at least one alteration in the promoter region, intron 1, intron 2, exon 3 and combinations thereof compared to the wild type SlMYB12 having the nucleic acid sequence set forth in of the SEQ ID NO:1.

According to certain embodiments, the detecting agent is a polynucleotide probe differentially hybridizable to the polynucleotide encoding the SlMYB12 variant compared to a wild-type SlMYB12 having the nucleic acid sequence as set forth in SEQ ID NO:1.

According to particular embodiments, the detecting agent is a polynucleotide probe differentially hybridizable to a polynucleotide having a nucleic acid sequence as set forth in any one of SEQ ID NOs:2-31 or part thereof compared to a wild-type SlMYB12 having the nucleic acid sequence set forth in SEQ ID NO:1.

According to other embodiments, the detecting agent is a primer pair capable of selectively amplifying the polynucleotide encoding the SlMYB12 variant compared to a wild-type SlMYB12 having the nucleic acid sequence as set forth in SEQ ID NO:1.

According to particular embodiments, the detecting agent is a primer pair capable of selectively amplifying SlMYB12 variant having a nucleic acid sequence as set forth in any one of SEQ ID NOs: 2-31 or part thereof compared to the wild type SlMYB12 having SEQ ID NO:1.

According to another aspect the present invention provides a method of screening for genetic markers indicative of the y mutant phenotype in a tomato plant, comprising (a) comparing the genomic polynucleotide sequence of the SlMYB12 gene having SEQ ID NO:1 of the wild type tomato plant or a fragment thereof to the genomic polynucleotide sequence of a SlMYB12 gene in a tissue sample obtained from a y phenotype tomato plant; (b) identifying alterations in the SlMYB12 genomic sequence of the y phenotype, wherein the alterations predict modification in the gene transcription and/or translation; wherein said alterations are genetic markers indicative of the y mutant phenotype.

For assay of genomic DNA, virtually sample from any plant tissue is suitable. For example, convenient samples include tissues obtained from roots, leaves, stem, and fruit and fruit parts. For assay of cDNA or mRNA, the tissue sample must be obtained from an organ in which the target nucleic acid is expressed. According to certain embodiments, the genomic DNA sample is obtained from leaves or roots. The sample may be further processed before the detecting step. For example, the DNA in the cell or tissue sample may be separated from other components of the sample, may be amplified, etc. All samples obtained from a plant, including those subjected to any sort of further processing are considered to be obtained from the plant.

In general, if the alteration is located in a gene, it may be located in a noncoding or coding region of the gene. If located in a coding region the alteration can result in an amino acid change. Such change may or may not have an effect on the function or activity of the encoded polypeptide. When the alteration is located in a non-coding region it can cause alternative splicing, which again, may or may not have an effect on the encoded protein activity or function.

In general, if the alteration is located in a gene, it may be located in a noncoding or coding region of the gene. If located in a coding region the alteration can result in an amino acid change. Such change may or may not have an effect on the function or activity of the encoded polypeptide. When the alteration is located in a non-coding region it can cause alternative splicing, which again, may or may not have an effect on the encoded protein activity or function. It should be understood that identifying markers associated with the y-phenotype by detecting a variant gene product(s) are also encompassed within the scope of the present invention. As used herein a “variant gene product” refers to a gene product which is encoded by an altered SlMYB12, including, but not limited to, a full length gene product, an essentially full-length gene product, a biologically active fragment of the gene product and a non-biologically non-active gene product. Biologically active fragments include any portion of the full-length polypeptide which initiates transcription comparable to the wild type.

A variant gene product is also intended to mean gene products which have altered expression levels or expression patterns which are caused, for example, by the variant allele of a regulatory sequence(s). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences).

According to certain embodiments, the alteration is identified in a non-coding region selected from the group consisting of an intron, a polyadenylation site and a leader sequence. According to other embodiments the alteration is identified in a regulatory sequence. According to certain currently preferred embodiments the alteration is identified in the promoter sequence.

Detection of alterations in the examined DNA typically requires amplification of the DNA taken from the candidate plant. Methods for DNA amplification are known to a person skilled in the art. Most commonly used method for DNA amplification is PCR (polymerase chain reaction; see, for example, PCR Basics: from background to Bench, Springer Verlag, 2000; Eckert et al., 1991. PCR Methods and Applications 1:17). Additional suitable amplification methods include the ligase chain reaction (LCR), transcription amplification and self-sustained sequence replication, and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Identification of the alteration in the SlMYB12 gene of the tomato y phenotype compared to the wild type gene can be performed by any method as is known to a person skilled in the art. According to certain embodiments, the alteration in the SlMYB12 sequence is determined by an assay selected from the group consisting of (a) observing shifts in electrophoretic mobility of single-stranded DNA on non-denaturing polyacrylamide gels; (b) hybridizing a SlMYB12 gene probe to genomic DNA isolated from the y phenotype tomato; (c) hybridizing an allele-specific probe to genomic DNA of said y phenotype tomato; (d) amplifying all or part of the SlMYB12 gene from said y phenotype tomato to produce an amplified sequence and sequencing the amplified sequence; (e) amplifying all or part of the SlMYB12 gene from said y phenotype tomato using primers for a specific SlMYB12 mutant allele; (f) molecularly cloning all or part of the SlMYB12 gene from said y phenotype tomato to produce a cloned sequence and sequencing the cloned sequence; (g) identifying a mismatch between (1) a SlMYB12 gene or a SlMYB12 mRNA isolated from said y phenotype tomato, and (2) a nucleic acid probe complementary to the tomato wild-type SlMYB12 gene sequence, when molecules (1) and (2) are hybridized to each other to form a duplex, (h) amplification of SlMYB12 gene sequences in said y phenotype tomato and hybridization of the amplified sequences to nucleic acid probes which comprise tomato wild-type SlMYB12 gene sequences, (i) amplification of SlMYB12 gene sequences in said tissue sample and hybridization of the amplified sequences to nucleic acid probes which comprise mutant SlMYB12 gene sequences, (j) screening for a deletion mutation in said y phenotype tomato, (k) screening for a point mutation in said y phenotype tomato, (l) screening for an insertion mutation in said y phenotype tomato, (m) in situ hybridization of the SlMYB12 gene of said y phenotype tomato with nucleic acid probes which comprise the SlMYB12 gene.

According to additional aspect, the present invention provides a method for identifying a tomato plant capable of producing fruit having the y phenotype comprising (a) providing a DNA sample from the plant before fruit are produced; (b) determining, in the DNA sample, the sequence of the SlMYB12 gene or part thereof (c) comparing the sequence to the sequence of a tomato wild type SlMYB12 gene; and (d) detecting at least one alteration in the SlMYB12 sequence from said sample, wherein the alteration is indicative of the capability of the plant to produce fruit having y phenotype.

According to one embodiment, the wild type SlMYB12 gene comprises a nucleic acid sequence as set forth in SEQ ID NO:1.

According to certain embodiments, the at least one sequence alteration results in down regulation of the expression or activity of SlMYB12.

According to other embodiments, the alteration in SEQ ID NO:1 is any one of those listed in Tables 1-3.

As described herein, the expression of the transcription factors SlMYB4-like and SlTHM27 was also down-regulated in fruit of the colorless peel y-phenotype. Furthermore, the expression was down regulated not only in fruit but also in other organs, including leaves. Thus, identifying tomato plants capable of producing fruit having the y phenotype by determining the expression level of SlMYB4-like and SlTHM27 is explicitly encompassed within the scope of the present invention. According to certain embodiments, the level of the SlMYB4-like and SlTHM27 transcripts is measured in the plant before fruit are produced. According to typical embodiments, the level is measured in a sample obtained from a plant leaf.

According to certain embodiments, identifying the at least one alteration is obtained by a technique selected from the group consisting of terminator sequencing, restriction digestion, allele-specific polymerase reaction, single-stranded conformational polymorphism analysis, genetic bit analysis, temperature gradient gel electrophoresis ligase chain reaction and ligase/polymerase genetic bit analysis.

According to other embodiments, the alteration in the SlMYB12 sequence is identified by employing nucleotides with a detectable characteristic selected from the group consisting of inherent mass, electric charge, electric spin, mass tag, radioactive isotope type bioluminescent molecule, chemiluminescent molecule, tagged nucleic acid molecule, hapten molecule, protein molecule, light scattering/phase shifting molecule and fluorescent molecule

The y Mutant Tissues Display Down-Regulation of Both Transcripts and Metabolites Associated with the Phenylpropanoid Pathway

The inventors of the present invention and co-workers had shown that the dramatic accumulation of flavonoids, one of the dominant classes of secondary metabolites in tomato fruit peel, pursues the formation of cuticular lipids and accelerates close to the breaker stage of fruit development (Mintz-Oron et al., 2008, ibid). The major differences in secondary metabolism between y and wt fruit tissues were observed at the orange stage and are mostly related to alterations in the phenylpropanoid/flavonoid pathway. Correlation between down-regulated transcripts and metabolites levels in this pathway was revealed along most of the biosynthetic pathway steps, including the SlPAL and Sl4CL genes and their related metabolites at the upper part of the pathway as well as SlCHS, SlCHI and NarCh/Nar and their derivatives.

The correlation between down-regulated transcripts and metabolites seemed weaker in the pathway branch leading to the biosynthesis of flavonols, in which significantly reduced expression levels of SlFLS in y peel led to the reduction of only one single flavonol species (i.e. quercetin-dihexose-deoxyhexose), while levels of all other detected flavonol derivatives did not differ from those of the wt. One possible explanation to this finding is the existence of additional low-abundant down-regulated flavonols, which for technical reasons were not identified in the present analysis. Another possible reason might be related to the relative early accumulation pattern of flavonols in the cv. Ailsa Craig (AC) as compared to other flavonoids (Mintz-Oron et al., 2008, ibid), so that flavonols might be less affected by the y mutation, which was most apparent in the breaker and orange stages of development. Unlike the y mutant (in the cv. AC background), the amiR-SlMYB12 transgenic plants (in the cv. MT background) exhibited a significant down-regulation in several flavonols (e.g. quercetin-hexose-deoxyhexose-pentose and quercetin-rutinoside (Rutin) in the fruit peel. This is possibly related to differences in flavonol accumulation between the two genetic backgrounds. The level of glycosylated metabolites along the pathway, such as coumaric-acid-hexose II and several NarCh- and/or Nar-hexoses, was also reduced. This might be due to a reduced expression of glycosyl transferases such as Sl3GT and SlRT.

In contrast to the overall down-regulation in gene expression and metabolite levels associated with the phenylpropanoid/flavonoid pathway, levels of some metabolites related to the lignin side-branch pathway were up-regulated in the y mutant, mostly the levels of ferulic acid derivatives. Albeit weaker, this effect was also significant in the y flesh tissue. However, this increase in metabolite levels that was evident in both tissue types was not accompanied by alterations in the gene expression. It is therefore suggested that the up-regulation of the lignin pathway branch results from a shift in the metabolic flux through the phenylpropanoid pathway rather than a direct outcome of changes in transcriptional regulation of this branch.

Unexpectedly, levels of the core phenylpropanoid precursor, the amino acid phenylalanine, was found to be significantly down regulated in the y-mutant only in the fruit flesh and not in the peel tissue. Without wishing to be bound by any specific theory or mechanism of action, this result might be explained by the more extensive usage of this phenylpropanoids amino acid precursor, which prevents its accumulation in the peel tissue. Alternatively, this might indicate that the major synthesis of the phenylalanine precursor takes places in the flesh, from where it is translocated to peripheral epidermal layers. Such tissue translocation of precursors and intermediates was previously suggested for phenylpropanoids, terpenoids and alkaloids, as well their biosynthetic intermediates, which are known to be synthesized in parenchymatic cells before their accumulation and storage in other tissues (Kutchan T. M. 2005. Curr. Opin. Plant Biol. 8: 292-300).

The Regulatory Network Controlling Flavonoid Accumulation in Tomato Fruit Peel

AtMYB12 was originally identified as a flavonol-specific transcription activator in Arabidopsis, and like its ortholog from maize (factor P) it does not require a bHLH partner for promoter activation (Mehrtens F. et al., 2005. Plant Physiol. 138: 1083-1096). Results published after the priority date of the present invention (Luo et al., 2008, ibid) suggested that the activity of AtMYB12 expressed in tomato mirrors the function of the tomato MYB12 orthologous protein, showing that AtMYB12 expressed in tomato activated flavonol biosynthesis as well as the caffeoylquinic acid (CQA) biosynthetic pathway. Levels of most identified flavonols were not significantly altered upon the down-regulation of SlMYB12 in the y phenotype plants, while targeted down-regulation of SlMYB12 in the 35S:amiR-SlMYB12 transgenic plants resulted in a significant reduction in flavonols levels. Without wishing to be bound by any specific theory or mechanism of action, this discrepancy is most likely due to the differences between cultivar AC (the y background) and cultivar MT (the amiR-SlMYB12 background). The effect of genetic background might be a result of variation in spatial and temporal expression of the flavonoid regulatory network that allows functional redundancy. Alternatively, it could be explained by polymorphism in promoters that alter target genes specificity.

While overexpression of AtMYB12 in tomato activated the CQA biosynthetic pathway, down-regulation of SlMYB12 in transgenic (cv. MT) plants also resulted in the accumulation of caffeic acid derivatives (e.g. dicaffeoylquinic acid III and tricaffeoylquinic acid). On the other hand, analysis of y phenotype (cv. AC background) did not reveal a significant change in the levels of most caffeic acid derivatives, besides the up-regulation in levels of caffeic acid hexose IV. Furthermore, levels of several ferulic acid derivatives in a closely related branch of the pathway were significantly increased in the y mutant. Therefore, the up-regulation of these related side branches (i.e. CQA and ferulic acid derivatives) upon both up and down regulation of MYB12 ortholog expression in tomato is more likely the result of a flux shift in the metabolic pathway rather than a direct outcome of altered transcriptional activation.

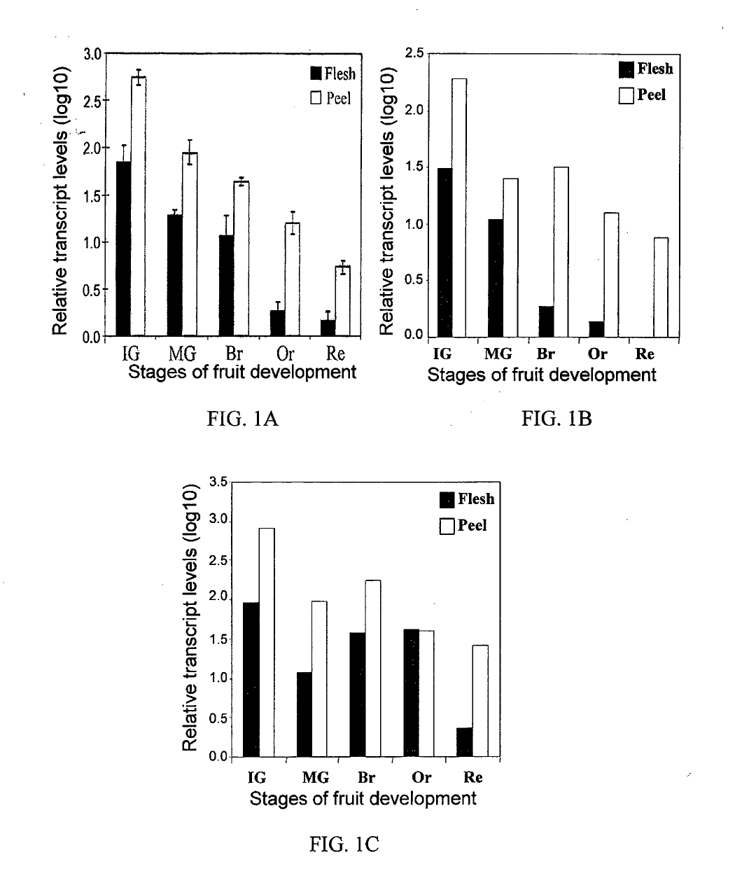

Stracke et al. (Stracke R. et al., 2007. Plant J. 50: 660-677) studied the functional redundancy and differential spatial expression characteristics of the R2R3-MYB factors subgroup 7 in Arabidopsis seedlings (AtMYB11, AtMYB12 and AtMYB111). They showed that all three members of this subgroup are flavonol-specific transcriptional regulators, and demonstrated that the final flavonol accumulation pattern is a result of the additive expression patterns of these three factors. Their results indicated that the detailed composition of flavonols that accumulate in different parts of the seedling depends on the differential (tissue- or cell-type-specific) responsiveness of target genes to the subgroup regulatory proteins. Furthermore, MYB11, MYB12 and MYB111 displayed very similar target gene specificity for several genes of flavonoid biosynthesis, including AtCHS, AtCHI, AtF3H and AtFLS. The present invention shows that in fruit of both the y-phenotype and the 35S:amiR-SlMYB12 plants, down-regulation of SlMYB12 is accompanied by the suppression of SlTHM27 (the tomato ortholog of AtMYB4) and SlMYB4-like, the complete sequence of which is provided herein for the first time. Both share a peel-associated expression profile during fruit development that is very similar to the pattern observed for SlMYB12 (FIG. 1). Without wishing to be bound by any specific theory or mechanism of action, these results suggest that SlMYB12 is likely to be a direct activator of the SlTHM27 and/or SlMYB4-like genes.

Flavonoids and other phenylpropanoids from plants are known to have a broad spectrum of health promoting effects, based mainly on their anti-oxidative activity. Dietary flavonoids were found to inhibit low-density lipid oxidation and thus reduce the risk to develop artherosclerosis and cardiovascular diseases. High consumption of flavonoids has also been shown to be associated with reduced risk to develop certain cancers and age-related degenerative diseases.

Thus, according to yet further aspect the present invention provides a transgenic plant comprising at least one cell comprising in its genome an exogenous polynucleotide encoding SlMYB12, wherein the plant has an elevated content of at least one phenylpropanoid selected from the group consisting of flavonoids, chlorogenic acid and derivatives thereof compared to a non-transgenic plant.

According to certain embodiment, SlMYB12 has an amino acid sequence as set forth in SEQ ID NO:33. According to other embodiments, the polynucleotide encoding SlMYB12 comprises a nucleic acid sequence as set forth in SEQ ID NO:1. According to additional embodiments the polynucleotide encoding SlMYB12 comprises a nucleic acid sequence as set forth in SEQ ID NO:32.

According to other embodiments, the flavonoids are flavonones and flavonols. According to other embodiments, the flavonoids are selected from the group consisting of naringenin, naringenin chalcone, eridictyol, phloretin, resveratrol, Quercetin-hexose-deoxyhexose-pentose (Q-triscch) and Quercetin rutinoside (Rutin).

Various DNA constructs may be used to obtain elevated amount of at least one phenylpropanoid in a plant cell according to the teachings of the present invention. The DNA construct or an expression vector comprising same may further comprise regulatory elements, including, but not limited to, a promoter, an enhancer, and a termination signal.

Among the most commonly used promoters are the nopaline synthase (NOS) promoter (Ebert et al., 1987 Proc. Natl. Acad. Sci. U.S.A. 84:5745-5749), the octapine synthase (OCS) promoter, caulimovirus promoters such as the cauliflower mosaic virus (CaMV) 19S promoter (Lawton et al., 1987 Plant Mol Biol. 9:315-324), the CaMV 35S promoter (Odell et al., 1985 Nature 313:810-812), and the figwort mosaic virus 35S promoter, the light inducible promoter from the small subunit of rubisco, the Adh promoter (Walker et al., 1987 Proc. Natl. Acad. Sci. U.S.A. 84:6624-66280, the sucrose synthase promoter (Yang et al., 1990 Proc. Natl. Acad. Sci. U.S.A. 87:4144-4148), the R gene complex promoter (Chandler et al., 1989 Plant Cell 1:1175-1183), the chlorophyll a/b binding protein gene promoter, etc. Other commonly used promoters are, the promoters for the potato tuber ADPGPP genes, the sucrose synthase promoter, the granule bound starch synthase promoter, the glutelin gene promoter, the maize waxy promoter, Brittle gene promoter, and Shrunken 2 promoter, the acid chitinase gene promoter, and the zein gene promoters (15 kD, 16 kD, 19 kD, 22 kD, and 27 kD; Perdersen et al. 1982 Cell 29:1015-1026). A plethora of promoters is described in International Patent Application Publication No. WO 00/18963.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht I L et al. (1989 Plant Cell 1:671-680).

Those skilled in the art will appreciate that the various components of the nucleic acid sequences and the transformation vectors described in the present invention are operatively linked, so as to result in expression of said nucleic acid or nucleic acid fragment. Techniques for operatively linking the components of the constructs and vectors of the present invention are well known to those skilled in the art. Such techniques include the use of linkers, such as synthetic linkers, for example including one or more restriction enzyme sites.

Methods for transforming a plant cell with nucleic acid sequences according to the present invention are known in the art. As used herein the term “transformation” or “transforming” describes a process by which a foreign DNA, such as a DNA construct, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to preferred embodiments the nucleic acid sequence of the present invention is stably transformed into a plant cell.

There are various methods of introducing foreign genes into both monocotyledonous and dicotyledonous plants (Potrykus I 1991 Annu Rev Plant Physiol Plant Mol Biol 42:205-225; Shimamoto K. et al., 1989. Nature 338:274-276).

The principal methods of the stable integration of exogenous DNA into plant genomic DNA include two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch et al., 1988. Plant Molecular Biology Manual A5, 1-9, Kluwer Academic Publishers, Dordrecht). A supplementary approach employs the Agrobacterium delivery system in combination with vacuum infiltration. The Agrobacterium system is especially useful in the generation of transgenic dicotyledenous plants.

Direct DNA uptake: There are various methods of direct DNA transfer into plant cells. In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

According to certain embodiments, transformation of the DNA constructs of the present invention into a plant cell is performed using Agrobacterium system.

The transgenic plant is then grown under conditions suitable for the expression of the recombinant DNA construct or constructs. Expression of the recombinant DNA construct or constructs alters the type and quantity of phenylpropanoids, particularly chlorogenic acid and flavonoids, including flavonones and flavonols in the transgenic plant compared to their quantity in a non transgenic plant.

The regeneration, development and cultivation of plants from single plant protoplast transformants or from various transformed explants is well known in the art (Weissbach and Weissbach, In.: Methods for Plant Molecular Biology, (Eds.), 1988 Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

Selection of transgenic plants transformed with a nucleic acid sequence of the present invention as to provide transgenic plants having altered amount of aromatic amino acids and secondary metabolites derived therefrom is performed employing standard methods of molecular genetic, known to a person of ordinary skill in the art. According to certain embodiments, the nucleic acid sequence further comprises a nucleic acid sequence encoding a product conferring resistance to antibiotic or herbicide, and thus transgenic plants are selected according to their resistance to the antibiotic or herbicide.

Extraction and detection of the metabolites synthesized by the transgenic plant cells can be performed by standard methods as are known to a person skilled in the art. According to certain embodiments, the metabolites of the present invention are extracted and analyzed by GC-MS as described by Mintz-Oron et al. (2008, ibid), LC-MS and HPLC as described by Fraser et al. 2000 (ibid).

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines, or pollen from plants of these important lines is used to pollinate regenerated plants. A transgenic plant of the present invention containing a desired metabolite is cultivated using methods well known to one of skill in the art.

There is a variety of methods in the art for the regeneration of plants from plant tissue. The particular method of regeneration will depend on the starting plant tissue and the particular plant species to be regenerated.

Also within the scope of this invention are seeds or plant parts obtained from the transgenic plants. Plant parts include differentiated and undifferentiated tissues, including but not limited to, roots, stems, shoots, leaves, pollen, seeds, tumor tissue, and various forms of cells and culture such as single cells, protoplasts, embryos, and callus

TABLE 4
Summary of phenylpropanoid/flavonoid related transcript
and their expression detected by arrays and RT-PCR

| | Gene | | | | | Real-Time | | |
|---|---|---|---|---|---|---|---|---|
| | Short | Gene | | Microarray | | PCR | | Real-Time |
| # | name-gene | Annotation | TC | Peel | Flesh | Peel | Flesh | Primers |
| 1 | CM | Chorismate Mutase | CK715539 | ↓ | [symbol] | | | |
| 2 | CM | Chorismate Mutase | TC174527 | [symbol] | [symbol] | | | |
| 3 | PDH | Prephenate | TC172766 | ↓ | [symbol] | ↓ | [symbol] | F; GAGTACATCGCCGCCAACA |
| | | Dehydratase | | | | | | (SEQ ID NO 40) |
| | | | | | | | | R; AGTCACGTTGCTTGAATCATCCT |
| | | | | | | | | (SEQ ID NO: 41) |
| 4 | PDH | Prephenate | TC180389 | [symbol] | [symbol] | | | |
| | | Dehydratase | | | | | | |
| 5 | PAR2 | Phenylacetaldehyde | BT013872.1 | ↑ | [symbol] | [symbol] | [symbol] | F; CCCTGGATGGAGCTAAGGAGA |
| | | Reductase | | | | | | (SEQ ID NO: 42) |
| | | | | | | | | R; CCTTCACACCCCTCAACAACA |
| | | | | | | | | (SEQ ID NO: 43) |
| 6 | PAL | Phenylalanine | TC170429 | ↓ | [symbol] | ↓ | [symbol] | F; CAGCCTAAGGAAGGACTTGCA |
| | | Ammonia-Lyase | | | | | | SEQ ID NO: 44) |
| | | | | | | | | R; GAAAATCGCTGACAAGACTTCAGA |
| | | | | | | | | (SEQ ID NO: 45) |
| 7 | PAL | Phenylalanine | TC172772 | ↓ | [symbol] | | | |
| | | Ammonia-Lyase | | | | | | |
| 8 | C4H | Cinnamate 4- | TC190665 | | | [symbol] | [symbol] | F; TCACGTCCACGTAACGTTGTG |
| | | Hydroxylase | | | | | | (SEQ ID NO: 46) |
| | | | | | | | | R; TGATACGTCTCATTTTTCTCCAATG |
| | | | | | | | | (SEQ ID NO: 47) |
| 9 | C3H | P-Coumaroyl 3′- | TC183733 | ↓ | [symbol] | ↓ | [symbol] | F; CACACTTTGGCTCGCAAACA |
| | | Hydroxylase | | | | | | (SEQ ID NO: 48) |
| | | | | | | | | R; CATATCCCATAGGAGGCCGATA |
| | | | | | | | | (SEQ ID NO: 49) |
| 10 | COMT1 | Caffeic Acid 3-O- | TC175188 | | | [symbol] | [symbol] | F; TTACCCTGGCGTTGAACACA |
| | | Methyltransferase | | | | | | (SEQ ID NO: 50) |
| | | | | | | | | R; TGCTCATCGCTCCAATCATG |
| | | | | | | | | (SEQ ID 51) |

tissue. The plant tissue may be in plant or in organ, tissue or cell culture.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Plant Material

Seeds from homozygous y plants (LA3189) in the cv. Ailsa Craig (AC) background as well as from wild type (wt) cv. AC were obtained from the Tomato Genetics Resource Center (TGRC; http://tgrc.ucdavis.edu). Flowers of greenhouse-grown plants were marked at anthesis, and fruit were harvested according to appearance and counted days post anthesis (DPA): ˜25 DPA—Immature Green, ˜42 DPA—Mature Green, ˜44 DPA—Breaker, ˜46 DPA—Orange and ˜48 DPA—Red. Each biological repeat was a mixture of four to five individual fruit from the same stage of development. Immediately upon harvesting, peel and flesh (without the gel and seeds) were manually dissected and frozen in liquid nitrogen.

### Flavonoids Detection

Diphenyl boric acid 2-amino-ethyl ester (DPBA, also called Naturstoff A) was used for the staining of free 3′, 4′ and/or 5′OH groups including un-glycosylated flavonoids. In initial analysis, untreated fruit slices and peel pieces were placed and photographed on a UV (312 mm) transilluminator. Subsequently, fruit slices and peel pieces were placed for 2 h in a saturated solution (<0.5% weight/vol) of DPBA (Sigma) with 0.01% triton X-100 and placed and photographed again on the UV transilluminator (Zerback R. et al., 1989. Plant Sci. 62: 83-91; Sheanan J. J. and Rechnitz G. A. 1992. BioTechniques 13: 880-883). Stained un-glycosylated flavonoids were colored in red while non-stained samples reflected only white illumination.

### Generation of Constructs and Plant Transformation

The full length SlCHS1 gene was isolated from genomic DNA of cv. AC fruit by PCR amplification using the primers listed in Table 4 and sub-cloned into pFLAP100 containing the CaMV 35S promoter and cloned into the binary pBINPLUS vector (Vanengelen F. A. et al., 1995. Transgenic Res. 4: 288-290). The amiR-MYB12 (artificial micro RNA-targeting MYB12) synthetic gene was synthesized by Bio S&T and cloned with CaMV 35S promoter into the pBINPLUS binary vector. Cotyledon transformation in cv. MicroTom tomato was performed according to Dan et al. (Dan Y. et al., 2006. Plant Cell Rep. 25: 432-441).

### Gene Expression Analysis

For array analysis total RNA was extracted by the hot phenol method (Verwoerd T. C. et al., 1989. Nucleic Acids Res. 17: 2362) from tissues of pooled 3-4 fruit from each of the three developmental stages examined. The cDNA synthesized by the Invitrogen Superscript II RTase was used as template to generate biotinylated cRNA that was fragmented and hybridized to the Affymetrix GeneChip® Tomato Genome Array as described in the Affymetrix technical manual (available at www.affymetrix.com), with two biological replicates for y and three biological replicates for wt. Replicate reproducibility and variance filtering procedures were carried out on wt transcripts expression data as previously described in Mintz-Oron et al. (2008, ibid). Normalization of log2-based expression intensity values was carried out using RMA analysis (Irizarry R. A. et al., 2003. Nucleic Acids Res. 31: e15) implemented by R microarray analysis package (http://www.R-project.org). Initial filtering of the genes was performed using the absent/present call acquired by MAS5 analysis software (Affymetrix 2002). Transcripts with at least one stage containing a present call were retained. Next, all expression values below the 10'Th percentile were set to the 10'th percentile value. Transcripts with poor quality spots showing low replicate reproducibility (high Relative Standard Deviation) in at least a third of the tested stages were eliminated from further analysis. Differential y mutant and wt transcripts were defined as those having at least two-fold intensity ratio in y vs. wt in at least one developmental stage of one tested fruit tissue (peel or flesh). Real time PCR analysis was carried out as described in Mintz-Oron et al. (2008). Summary of phenylpropanoid/flavonoid related transcript and their expression detected by arrays and RT-PCR, as well as the primer sequences designed by the Express software (Applied Biosystems) are provided in Table 4 hereinbelow.

Gene

| | | |
|---|---|---|
| Short | Micro- | Real-Time |

| | name- | Gene | | array | | PCR | | Real-Time |
|---|---|---|---|---|---|---|---|---|
| # | gene | Annotation | TC | Peel | Flesh | Peel | Flesh | Primers |
| 11 | COMT2 | Caffeic Acid 3-O- | TC177389 | | | [symbol] | [symbol] | F; TGACCTACCCAATGTCATCAAAGAT |
| | | Methyltransferase | | | | | | (SEQ ID NO: 52) |
| | | | | | | | | R; GAATGATTAGCTCCCCTTGAGGA |
| | | | | | | | | (SEQ ID NO: 53) |
| 12 | 4CL | 4-Coumarate CoA | TC173193 | ↓ | [symbol] | ↓ | ↓ | F; AACCCCACTGCTAAGGCTATM |
| | | Ligase | | | | | | (SEQ ID NO: 54) |
| | | | | | | | | R; GACAATTACCCCCAAATGTCCTAA |
| | | | | | | | | (SEQ ID NO: 55) |
| 13 | 4CL | 4-Coumarate CoA | TC173154 | [symbol] | ↓ | | | |
| | | Ligase | | | | | | |
| 14 | 4CL | 4-Coumarate CoA | TC176157 | [symbol] | [symbol] | | | |
| | | Ligase | | | | | | |
| 15 | CHS1 | Chalcone Synthase | TC170658 | ↓ | ↓ | ↓ | ↓ | F; TGGTCACCGTGGAGGAGTATC |
| | | | | | | | | (SEQ ID NO: 56) |
| | | | | | | | | R; GATCGTAGCTGGACCCTCTGC |
| | | | | | | | | (SEQ ID NO: 57) |
| | | | | | | | | F*; GCATATCCACCATTTTTTCCGGC |
| | | | | | | | | (SEQ ID NO: 58) |
| | | | | | | | | R*; CCCACAATGTAAGCCCAGCCC |
| | | | | | | | | (SEQ ID NO: 59) |
| 16 | CCR | Cinnamoyl CoA | TC180112 | ↓ | ↓ | ↓ | ↓ | F; CACGGAACGAATGGCATTTA |
| | | Reductase-Like | | | | | | (SEQ ID NO: 60) |
| | | | | | | | | R; TTCCAGATGCATGCAAGTAGAGA |
| | | | | | | | | (SEQ ID NO: 61) |
| 17 | CCR | Cinnamoyl CoA | TC178150 | ↓ | [symbol] | | | |
| | | Reductase-Like | | | | | | |
| 18 | CCR | Cinnamoyl CoA | TC170723 | ↓ | [symbol] | | | |
| | | Reductase | | | | | | |
| 19 | REF1 | Reduced Epidermal | TC181878 | [symbol] | [symbol] | [symbol] | [symbol] | F; TTGGCGATCCCTTCAAGAAA |
| | | Fluorescence | | | | | | (SEQ ID NO: 62) |
| | | | | | | | | R; AGAGCTGTCACGGCCTTCTC |
| | | | | | | | | (SEQ ID NO: 63) |
| 20 | CHI | Chalcone Isomerase/ | TC178705 | ↓ | ↓ | ↓ | ↓ | F; CCCTTGTTCCACCTAAGTACCATT |
| | | chalcone-flavanone | | | | | | (SEQ ID NO: 64) |
| | | isomerase | | | | | | R; GTGCTTCTGGGAGTGCAAAGA |
| | | | | | | | | (SEQ ID NO: 65) |
| 21 | CHI | Chalcone Isomerase | TC177570 | | | | | |
| 22 | CHI | Chalcone Isomerase | NP840677 | | | | | |
| 23 | F3H | Flavanone 3- | TC180957 | ↓ | ↓ | ↓ | ↓ | F; CTGTTCAGCCCGTTGAAGGT |
| | | Hydroxylase | | | | | | (SEQ ID NO: 66) |
| | | | | | | | | R; ACCACTGCTTGATGATCAGCAT |
| | | | | | | | | (SEQ ID NO: 67) |
| 24 | F3H | Flavanone 3- | TC181836 | ↑ | [symbol] | | | |
| | | Hydroxylase | | | | | | |
| 25 | F3H | Flavanone 3- | TC178533 | [symbol] | [symbol] | | | |
| | | Hydroxylase | | | | | | |
| 26 | F3′H-like | Flavonoid 3′- | TC175149 | | | | | F; ATTCGCCGACGGTACTAACG |
| | | Hydroxylase-Like | | | | | | (SEQ ID NO: 68) |
| | | | | | | | | R; ATCGCCGATGTTGAAAACG |
| | | | | | | | | (SEQ ID NO: 69) |
| 27 | FLS | Flavonol Synthase | TC172800 | ↓ | [symbol] | ↓ | ↓ | F; GAGCATGAAGTTGGGCCAAT |
| | | | | | | | | (SEQ ID NO: 70) |
| | | | | | | | | R; TGGTGGGTTGGCCTCATTAA |
| | | | | | | | | (SEQ ID NO: 71) |
| 28 | ANS-like | Anthocyanidin | TC175220 | | | ↓ | ↓ | F; TTGGTTTGGAAGGCCATGAA |
| | | Synthase-Like | | | | | | (SEQ ID NO: 72) |
| | | | | | | | | R; AAATCAGGCCTTGGACATGGT |
| | | | | | | | | (SEQ ID NO: 73) |
| 29 | 3GT | Flavonoid 3-Glucosyl | TC176277 | ↓ | ↓ | [symbol] | [symbol] | F; TCACAAGCCTACTTAATTTGTTCCA |
| | | Transferase | | | | | | (SEQ ID NO: 74) |
| | | | | | | | | R; GCTCGAGGGAAAGTTCTAGATGAA |
| | | | | | | | | (SEQ ID NO: 75) |
| 30 | 3GT | Flavonoid 3-Glucosyl | TC176549 | ↓ | [symbol] | [symbol] | [symbol] | F; TGGGATGGCGTCAAACAAG |
| | | Transferase | | | | | | (SEQ ID NO: 76) |
| | | | | | | | | R;CCCTGTTICCTCCTCTGCTTCT |
| | | | | | | | | (SEQ ID 77) |
| 31 | RT | Rhamnosyl Transferase | TC179039 | ↓ | [symbol] | ↓ | [symbol] | F; TGCAGGATTCAGTTCAGTGATAGAG |
| | | | | | | | | (SEQ ID NO: 78) |
| | | | | | | | | R; TCATATCCCCACTCACTAGTMGC |
| | | | | | | | | (SEQ ID NO: 79) |
| 32 | S1_JAF13 | S1_JAF13 | TC182581 + | | | [symbol] | [symbol] | F; GCAATCTTCTGGTCAACTGCAG |
| | | | TC190452 + | | | | | (SEQ ID NO: 80) |
| | | | TC185386 + | | | | | R; CCCGCCTGAACAGTCTTCC |
| | | | TC178931 | [symbol] | [symbol] | | | (SEQ ID NO: 81) |
| | | | | | | | | F*; GAATATATGCCAAGTTGTAGCAAGTC |
| | | | | | | | | (SEQ ID NO: 82) |
| | | | | | | | | R*; CACAAAAAAGTGATGATCATGAAAG |
| | | | | | | | | (SEQ ID NO: 83) |
| 33 | S1_MYB12- | S1_MYB12-like | AI771790 | | | [symbol] | [symbol] | F; CCAAACGAGGACGCAGTAGAA |
| | like | | | | | | | (SEQ ID NO: 84) |
| | | | | | | | | R; ATGCCATAACATCTGGTCATCAAT |
| | | | | | | | | (SEQ ID NO: 85) |
| 34 | S1_MYB12 | S1_MYB12 | TC172990 | | | ↓ | [symbol] | F; GCCAGCTTGTGATAGTGCCAT |
| | | | | | | | | (SEQ ID NO: 86) |
| | | | | | | | | R; AGGGCTTCCCTTGGCTTCTA |
| | | | | | | | | (SEQ ID NO: 87) |
| | | | | | | | | F*: ATGGGAAGAACACCTTGTTGT |
| | | | | | | | | (SEQ ID NO: 88) |
| | | | | | | | | R*: TCAAAAGCAATATATAATGTCATA |
| | | | | | | | | (SEQ ID NO: 89) |
| 35 | S1_MYB4- | S1_MYB4-like | TC184379 | ↓ | [symbol] | ↓ | [symbol] | F; AGGGCTTCCCTTGGCTTCTA |
| | like | | | | | | | (SEQ ID NO: 90) |
| | | | | | | | | R; ATTGATGAGGCGTTGGTCTTCTT |
| | | | | | | | | (SEQ ID NO: 91) |
| | | | | | | | | F*; CAAAGTATGGGACGTTCACC |
| | | | | | | | | (SEQ ID NO: 92) |
| | | | | | | | | R*; CCACCATGATATCCATTTGC |
| | | | | | | | | (SEQ ID NO: 93) |
| 36 | THM27 | THM27 | TC174616 | ↓ | [symbol] | ↓ | [symbol] | F; GTAAAGATTGCAGTTGTGGAAGTGA |
| | | | | | | | | (SEQ ID NO: 94) |
| | | | | | | | | R; TTCAAGCCCAAAAAGTCATAACC |
| | | | | | | | | (SEQ ID NO: 95) |
| | | | | | | | | F*; CCATTATCCTTCTCTCAATTGG |
| | | | | | | | | (SEQ ID NO: 96) |
| | | | | | | | | R*; CTATATTGCAAAGTTTACAACCATG |
| | | | | | | | | (SEQ ID NO: 97) |
| 37 | S1_ANT2 | S1_ANT2 | TC186580 | | | [symbol] | [symbol] | F; CCAGGAAGGACAGCAAACGA |
| | | | | | | | | (SEQ ID NO: 98) |
| | | | | | | | | R;CGAGGACGAGAATGAGGATGTAG |
| | | | | | | | | (SEQ ID NO: 99) |
| | | | | | | | | F*; GAATACTCCTATGTGTGCATC |
| | | | | | | | | (SEQ ID NO: 100) |
| | | | | | | | | R*; CAAAAATAAAAATTCTTTAATT |
| | | | | | | | | AAGT (SEQ ID NO: 101) |
| 38 | S1_MYB111 | S1_MYB111 | TC178481 | | | [symbol] | [symbol] | F; TCCTGATCTCAAACATGGGAAAAT |
| | | | | | | | | (SEQ ED NO: 102) |
| | | | | | | | | R; TTTTTCGGGCCATTCTTGAC |
| | | | | | | | | (SEQ ID NO: 103) |
| | | | | | | | | F*; GATGGTGCAAGAAGAAATAATGAG |
| | | | | | | | | (SEQ ID NO: 104) |
| | | | | | | | | R*; CGATAGCGAAAATATGTCACATTG |
| | | | | | | | | (SEQ ID NO: 105) |
| 39 | S1_MYB61 | S1_MYB61 | AW626100 + | | | [symbol] | [symbol] | F; TGGCTGTTGGAGCTCTGTCC |
| | | | TC183887 | | | | | (SEQ ID NO: 106) |
| | | | | | | | | R; CTCTTTTCAAATCAGGCCTCAAG |
| | | | | | | | | (SEQ ID NO: 107) |
| | | | | | | | | F*; GGCCGGGAGAGGCTTTTAT |
| | | | | | | | | (SEQ ID NO: 108) |
| | | | | | | | | R*; CTATCATATACCATCCACAAAAG |
| | | | | | | | | (SEQ ID NO: 109) |

### Non-Targeted UPLC-QTOF-MS Profiling of Semi-Polar Compounds and Data Analysis

Non-targeted analysis of semi-polar compounds was carried out by the UPLC-QTOF instrument (Waters, Premier), with the UPLC column connected online to a UV detector and then to the MS detector as previously described (Mintz-Oron et al., 2008, ibid). Separation of metabolites was performed with the gradient elution (acetonitrile-water, containing 0.1% formic acid) on the 100×2.1-mm i.d., 1.7-μm UPLC BEH C18 column (Waters Acquity). Masses of the eluted compounds (m/z range from 50 to 1,500 Da) were detected with a QTOF MS equipped with an ESI source (performed in both positive and negative modes). XCMS data processing (Smith C. A. et al., 2006. Anal Chem 78: 779-787) was carried out as previously described by Mintz-Oron et al., (2008, ibid).

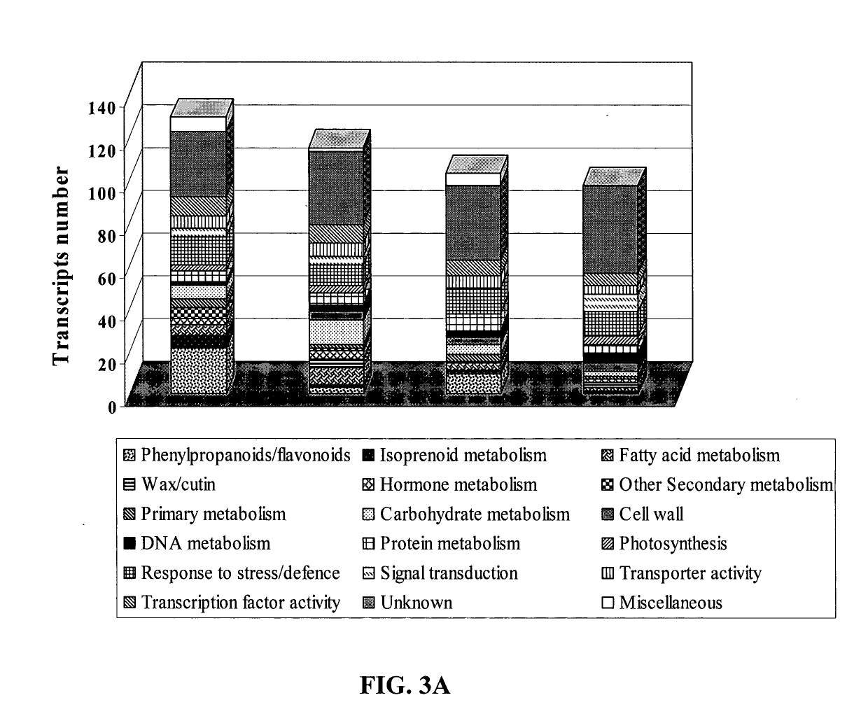

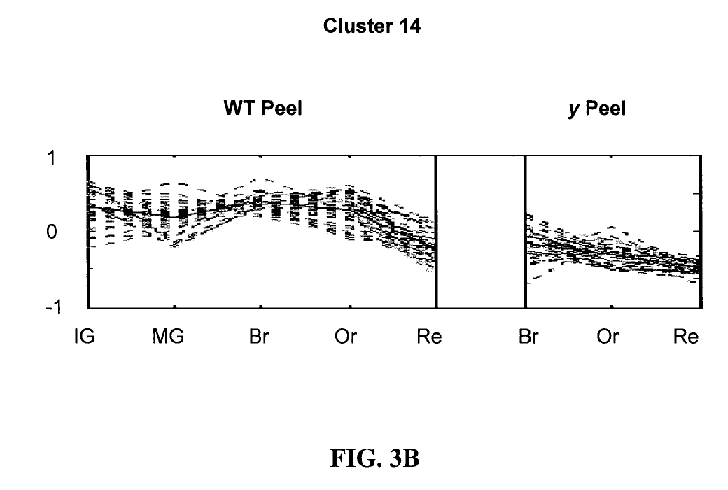

Principal Component Analysis (PCA) of metabolic profiles was performed on data sets obtained as XCMS output with the MATLAB Statistical Toolbox. The PCA plot presented in FIG. 3 was constructed with the software package TMEV (Saeed A. I. et al., 2003. BioTechniques 34: 374-378), the data were pretreated by normalization to the median of the entire sample set for each mass signal and log₁₀ transformation. Metabolites that differed between y and wt fruit tissues were detected in the breaker, orange and red developmental stages. To ensure data robustness to statistical analysis, mass signals for which the maximal intensity across all samples was <50 units for peel samples or <40 units for flesh samples (arbitrary units proportional to peak area calculated by XCMS) were discarded. Statistical filtering was carried out on the mass signals to identify differential markers between the wt and y mutant. The remaining peaks were those positive for the following filter in at least one of the three developmental stages:

$$\vert {log}_{2} ( \frac{\overline{WT}}{\overline{mut}} ) \vert - max [ {log}_{2} ( \frac{max ( WT )}{min ( WY )} ) , {log}_{2} ( \frac{max ( mut )}{min ( mut )} ) ] \geq 1$$

i.e. the fold change between the means of wt and mutant had to be at least twice higher than the maximal fold change within the repeats of either y mutant or wt. Mass signals of interest retained after the filtering stage were assigned to metabolites using automatic assignment of all mass signals to metabolites by clustering. To cluster together masses belonging to the same metabolite, a custom computer program implemented in MATLAB 7.3 was developed. The program accepts as an input the filtered intensity data following XCMS analysis and the chromatographic retention time of each marker. A matrix is calculated that describes the distance between all pairs of mass signals based on Spearman correlation among their intensities across all samples and difference in their retention times. Therefore: distance Dij between two markers i and j with retention times RTi and RTj and Spearman correlation coefficient ρij between their intensities across all samples was defined as:

Dij=1−ρij if |RTi−RTj|<3 sec,

Else:

Dij=100*|RTi−RTj|

A hierarchical average linkage clustering algorithm was applied to the distance matrix to define mass-signals to metabolite assignments. The clustering distance cutoff was set to 0.34. The cutoff was determined by maximization of the similarity assessed by the Jaccard similarity coefficient of the clustering results to the test set containing manual assignment of mass signals to 26 metabolites. Jaccard similarity coefficient is defined as:

$$Jaccard = \frac{n 11}{n 11 + n 10 + n 01}$$

Where for each pair of mass signals from the automatically clustered set and the manually curated assignment set:

n11 is the amount of pairs that were assigned to the same metabolite both automatically and manually;

n10 is the amount of pairs that were assigned to the same metabolite manually, but not automatically; and

n01 is the amount of pairs that were assigned to the same metabolite automatically, but do not belong to the same metabolite in the manual assignment.

MS/MS was performed on manually selected molecular ions of differential metabolites with high intensity. The intensity values for these compounds resulting from XCMS analysis were manually checked and reintegrated in cases of problematic peaks.

### GC-MS Profiling of Derivatized Polar Extracts and Data Analysis

GC-MS analysis of polar metabolites in y mutant and wt fruit tissues samples (n=3) was carried out as previously described in Mintz-Oron et al. (2008, ibid). For PCA plotting, the data was pretreated as follows: missing values for metabolites in one of the three replicates were exchanged for the average between the replicates, zero values were replaced by value 10 times lower than the minimal non-zero value in the dataset, data were normalized to the mean of each metabolite across all samples and log transformed. For statistical analysis of the whole data matrix a two-way ANOVA test was performed with the two discriminating factors being the genotype (either wt or y) and the fruit development stage. Multiple hypothesis control was carried out by an FDR procedure.

### Example 1

### Microarray Analysis of Transcriptional Changes in Flesh and Peel Tissues of the y Mutant Compared to Wild Type

Transcriptome analysis was carried out in order to compare gene expression in y and wt fruit (either peel or flesh tissues) at three developmental stages (breaker, orange and red). A total of 406 non-redundant transcripts exhibited a two-fold or higher increased or decreased expression in the y mutant vs. wt peel or flesh in at least one of the three tested stages of fruit development. Most of these transcripts (353) differed at only one developmental stage, 56 transcripts differed at two developmental stages, and 16 differed at all three developmental stages. Sixty of these 406 transcripts differed in both flesh and peel tissues. The differences in expression of 17 selected transcripts (putatively identified as tomato PDH, PAL, C3H, 4CL, CHS1, CHS2, CCR, CHI, F3H, FLS, RT, C3H, CCR, THM27, MYB4-like, SlNCED and SlCRTR-B2) were confirmed by means of quantitative Real-Time (RT) PCR analyses.

The differential transcripts were assigned to putative functional categories based on sequence similarities to studied homologue/ortholog from other species (FIG. 3A). The most represented functional category included 21 phenylpropanoid/flavonoid-related transcripts that were down-regulated in the y mutant fruit peel; nine of these transcripts were down-regulated in the flesh as well. Six transcripts putatively associated with isoprenoid metabolism were also down-regulated in y peel. On the other hand, two groups of putative carbohydrate and fatty acid metabolism related transcripts (11 and 8, respectively) were up-regulated in y peel. Various transcription factors were up- or down-regulated in y mutant tissues, among which were two members of the R2-R3 MYB family, SlTHM27 and SlMYB4-like (TC174616 and TC184379, respectively), i.e. tomato homologues of the AtMYB4 flavonoid related transcription factor, that were down-regulated in both peel and flesh tissues of the y mutant.

In order to study the expression patterns of genes differing between y and wt during fruit development, all 406 transcripts with a two-fold and more difference between the two genotypes were clustered (in at least one developmental stage, either in peel or in flesh). Forty expression profile clusters were created, 20 for flesh and 20 for peel. Cluster #14, for example, is composed of 38 transcripts that exhibited lower expression in the y peel (FIG. 3B). These peel down-regulated genes included 15 transcripts putatively related to phenylpropanoid/flavonoids metabolism, including 2 transcription factors (SlTHM27 and SlMYB4like), 6 transcripts associated with response to stress and defense, 2 transcripts related to fatty acid metabolism and 15 transcripts from other categories. Eleven transcripts belonging to this cluster (including 8 phenylpropanoid/flavonoid-related) were down-regulated in the y peel at all three developmental stages (Table 5).

TABLE 5
Genes down/up- regulated in peel or flesh tissues of the y mutant at the
three tested stages of tomato fruit development (breaker, orange, red)

| | | | |
|---|---|---|---|
| Identifier | Gene annotation^{a} | Pathway | Cluster^{b} |

Down-regulated in y fruit peel at the three tested developmental stages

| | | | |
|---|---|---|---|
| BI209975^{c} | Lipase | Fatty acid metabolism | 14 |
| TC178705 | Chalcone isomerase (CHI) | Phenylpropanoids/flavonoids | 14 |
| TC180957 | Flavanone 3-hydroxylase (F3H) | Phenylpropanoids/flavonoids | 14 |
| TC176277 | Flavonoid 3-glucosyl transferase | Phenylpropanoids/flavonoids | 14 |
| | (3GT) | | |
| TC170658 | Chalcone synthase (CHS1) | Phenylpropanoids/flavonoids | 14 |
| TC170429 | Phenylalanine ammonia-lyase | Phenylpropanoids/flavonoids | 14 |
| | (PAL) | | |
| TC179039 | Rhamnosyltransferase (RT) | Phenylpropanoids/flavonoids | 14 |
| TC180112 | Cinnamoyl CoA reductase-like | Phenylpropanoids/flavonoids | 14 |
| | (CCR) | | |
| TC176549 | Flavonoid 3-glucosyl transferase | Phenylpropanoids/flavonoids | 14 |
| | (3GT) | | |
| TC186636 | C-4 sterol methyl oxidase (SMO) | Isoprenoid | 14 |
| TC178916 | Putative glycine-rich RNA | Unknown | 17 |
| | binding protein | | |
| TC187382 | Unknown | Unknown | 14 |

Up-regulated in y fruit peel at the three tested developmental stages

| | | | |
|---|---|---|---|
| AW039066 | Lipase (EXL1) | fatty acid metabolism | 5 |
| TC177136 | Annexin | Unknown | 20 |
| TC173084 | Unknown | Unknown | 20 |

Down-regulated in y fruit flesh at the three tested developmental stages

| | | | |
|---|---|---|---|
| TC171069 | Unknown | Unknown | 11 |

Up-regulated in y fruit flesh at the three tested developmental stages

| | | | |
|---|---|---|---|
| TC177136 | Annexin | Unknown | 14 |

^{a}Putative annotation of transcripts and pathways are based on the closest known homologues/ortholog from other species.
^{b}Peel/flesh expression profile clusters to which the gene belongs.
^{c}GB accessions are given when no TC index (TIGR identifier) is available.

### Example 2

### Both Primary and Secondary Metabolism are Affected in the Developing y Mutant Fruit

A comprehensive metabolome analyses of y and wt tissues during 5 stages of fruit development (immature green, mature green, breaker, orange and red) was performed n order to examine the effect of the y lesion on fruit metabolism. Various analytical methods were employed including: Ultra Performance Liquid Chromatography coupled to a Quadrupole Time-Of-Flight Mass Spectrometry (UPLC-QTOF-MS) for the detection of semi-polar components; Gas Chromatography-MS (GC-MS) analysis for the identification of polar compounds; GC with flame ionization detector (GC-FID) for the profiling of waxes in isolated fruit cuticles; and HPLC coupled to UV and fluorescence detectors for the analysis of lipid-soluble isoprenoids.

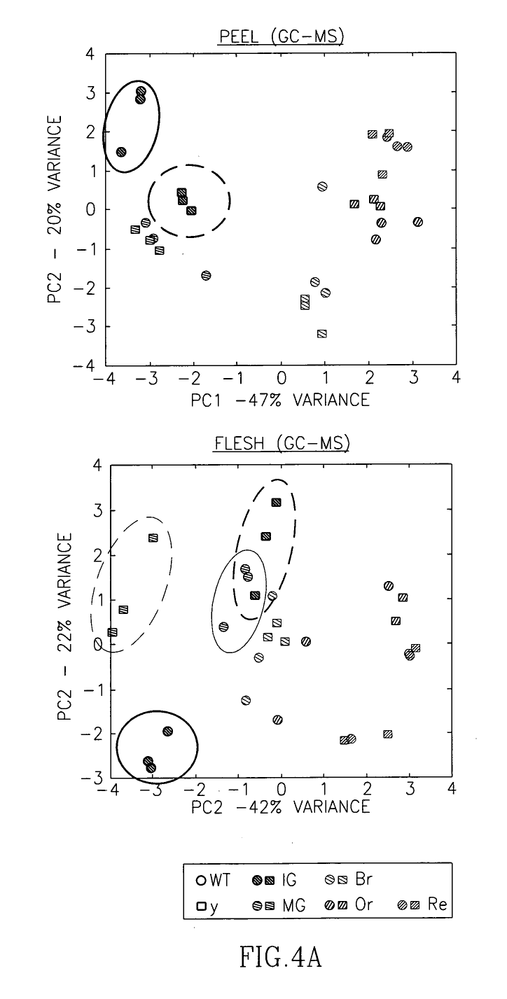

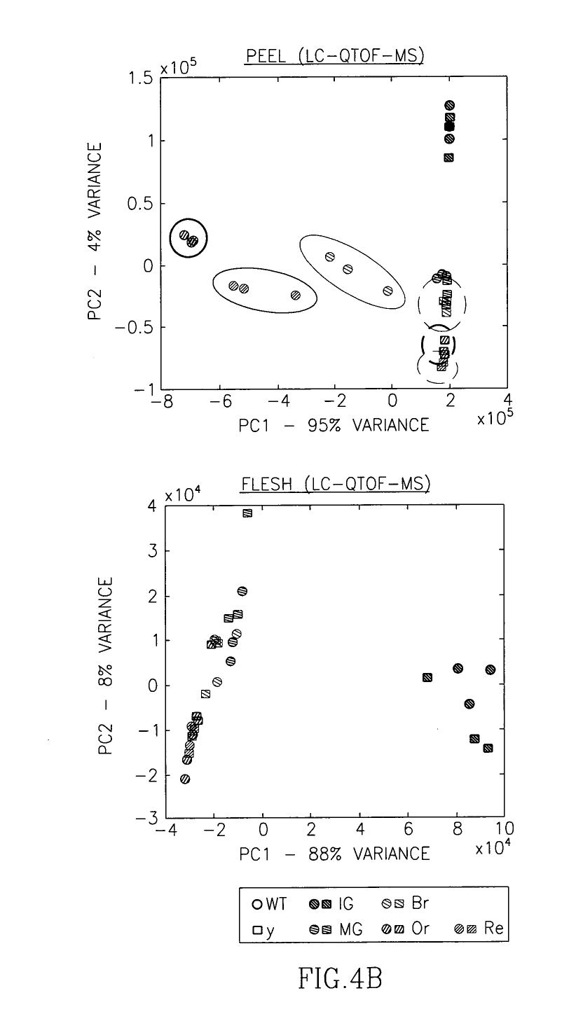

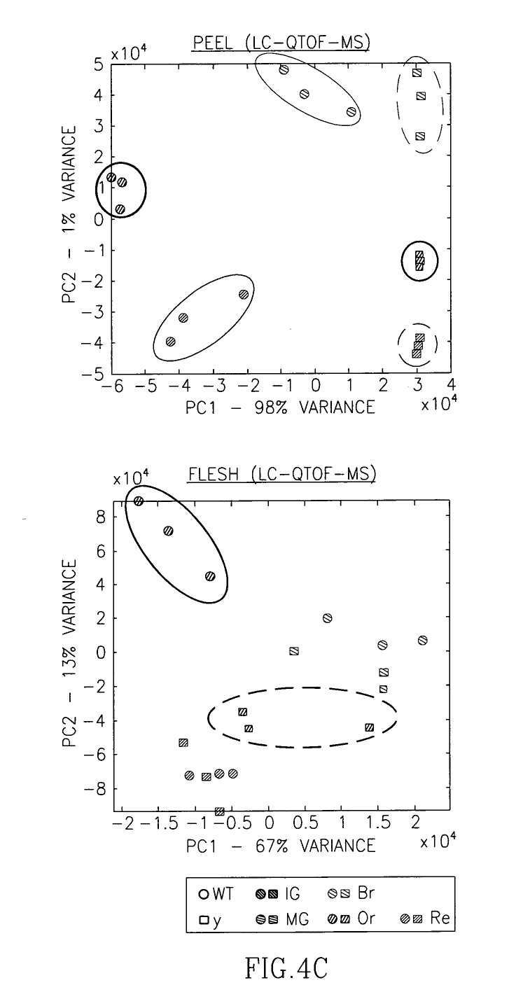

To obtain a general view on the differences in metabolite profiles between y and wt fruit tissues, a Principal Component Analysis (PCA) with the metabolite GC-MS and LC-MS data sets was conducted (FIG. 4). In the GC-MS set, PCA could distinguish between the metabolite profiles of y and wt fruit only at early stages of development, i.e. at the immature green stage in the peel and at the immature and mature green stages in the flesh (FIG. 4A). In contrast, PCA of the UPLC-QTOF-MS data set (negative ESI mode), derived from the peel tissue, could distinguish the y and wt metabolite profiles at the three latest stages of fruit development. Such differences were not evident in the case of the flesh tissue samples when all 5 tested stages of fruit development were analyzed together (FIG. 4B). However, when PCA was carried out on a dataset that excluded the two early stages (immature and mature green), a clear distinction was also demonstrated between the metabolic profiles of y and wt in peel samples of the three late stages, as well as in flesh samples at the orange stage (FIG. 4C).

Thus, at early stages of fruit development y and wt retain different metabolic profiles that are mainly due to changes in levels of polar (mostly primary) metabolites detected by GC-MS analysis. Differences between y and wt fruit in secondary metabolites (mostly detected by UPLC-QTOF-MS) appear at later stages of fruit development, predominantly in the peel tissue.

### Example 3

### Levels of Primary Metabolites in y Fruit at Early Stages of Development

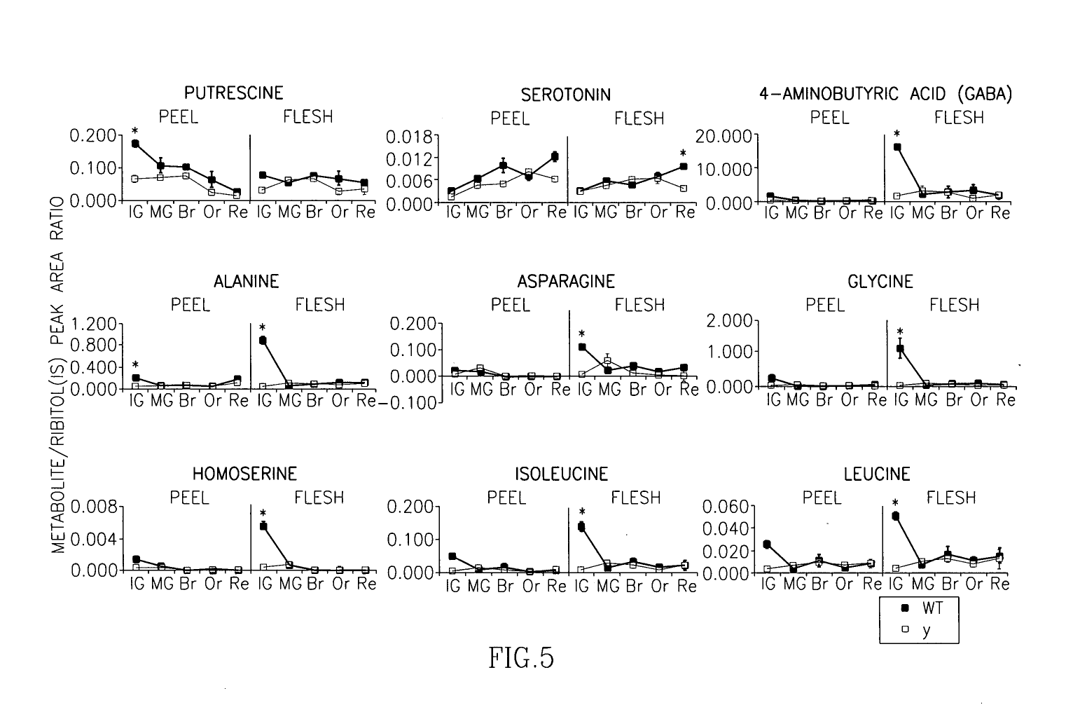

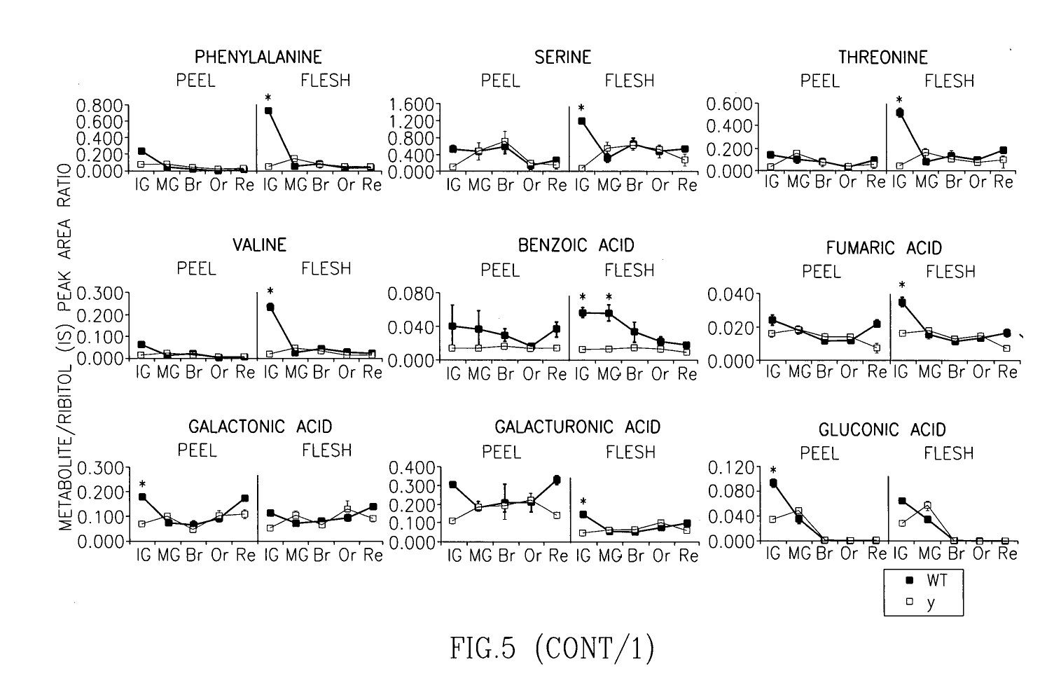

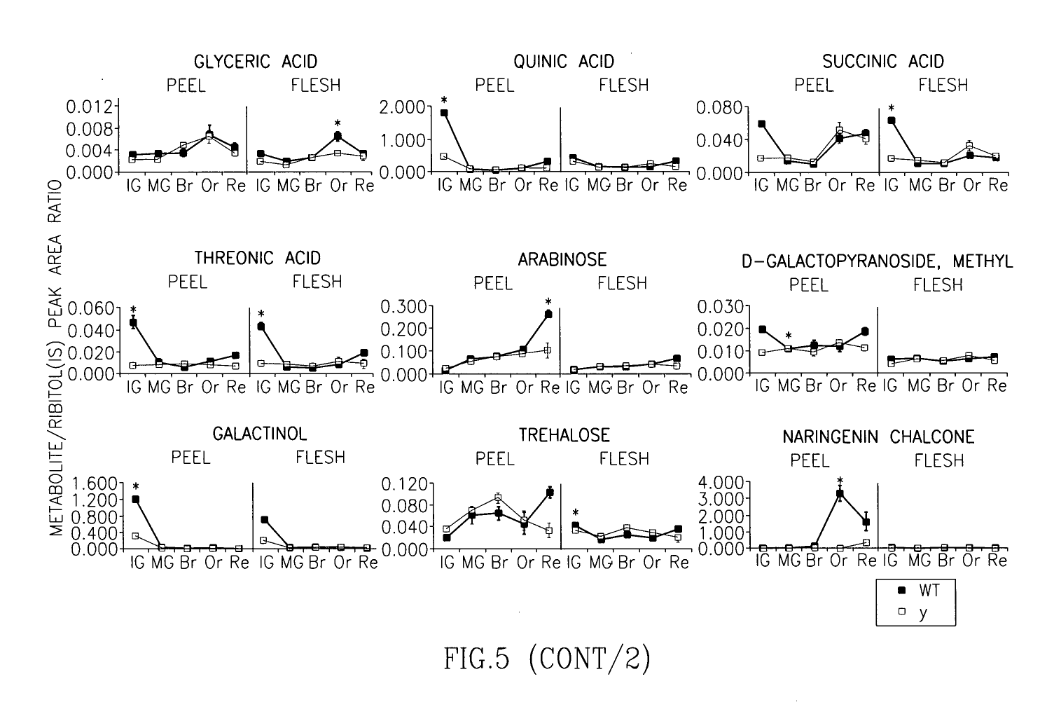

Out of the 56 metabolites that could be monitored by the GC-MS technology, most of which were primary metabolites, 27 (including two amines, eleven amino acids, nine organic acids, four sugars and NarCh) significantly differed between y and wt tissues in at least one stage of fruit development (all exhibited reduced levels in y fruit tissues; FIG. 5). The majority of the differential metabolites (23 out of 27) showed reduced levels in the immature green stage of y fruit. The other four differential metabolites, including NarCh, arabinose, glyceric acid and the amine serotonin, significantly differed at the orange and red stages. Four differential metabolites, i.e. 4-aminobutyric acid (GABA), alanine, valine and threonic acid, were significantly different between y and wt fruit in both peel and flesh tissues, 15 were significantly different only in the flesh tissue and eight were different only in the peel tissue. Interestingly, two phenylpropanoid precursors, phenylalanine and benzoic acid, significantly differed between y and wt in immature green fruit, but only in the flesh tissue. To summarize, levels of various primary metabolites, particularly amino acids and organic acids, are reduced in early y fruit development mostly in the flesh tissue.

### Example 4

### Alterations in Gene Expression and Metabolism Associated with the Phenylpropanoid and Flavonoid Pathways in the y Mutant

UPLC-QTOF-MS metabolite analysis resulted in the assignment of 71 putative metabolites, mostly secondary metabolites, in developing tomato fruit tissues. According to a two-way ANOVA test, twenty-nine of these metabolites significantly differed between y and wt fruit peel tissues (Table 6). Only nine of these metabolites were significantly altered in fruit flesh tissues (Table 7). All differential metabolites were products of the phenylpropanoid and flavonoid pathways

TABLE 6
UPLC-QTOF-MS detected metabolites that are differentially
produced between y mutant and wt fruit peel tissues

| | | | |
|---|---|---|---|
| Peak | Putative | Molecular | Differential |
| No.¹ | metabolite | formula | stage |

Metabolites down-regulated in the y mutant peel

| | | | |
|---|---|---|---|
| 59 | Hydrocinnamic acid-hexose | C₁₅H₂₀O₈ | Br, Or, Re⁵ |
| 39 | Coumaric acid-hexose II | C₁₅H₁₈O₈ | Or |
| 42 | trans-Resveratrol (S) | C₁₄H₁₂O₃ | Br, Or, Re |
| 23 | NarCh² (S)³ | C₁₅H₁₂O₅ | Br, Or, Re |
| 25 | NarCh-hexose I | C₂₁H₂₂O₁₀ | Or, Re |
| 26 | NarCh-hexose II | C₂₁H₂₂O₁₀ | Or, Re |
| 27 | NarCh-hexose III | C₂₁H₂₂O₁₀ | Or, Re |
| 24 | NarCh-dihexose | C₂₇H₃₂O₁₅ | Or, Re |
| 35 | Hydroxylated NC | C₁₅H₁₂O₆ | Br, Or, Re |
| 22 | Nar⁴ (S) | C₁₅H₁₂O₅ | Or, Re |
| 28 | Nar-hexose | C₂₁H₂₂O₁₀ | Or |
| 29 | Nar-dihexose I | C₂₇H₃₂O₁₅ | Or, Re |
| 30 | Nar-dihexose II | C₂₇H₃₂O₁₅ | Or, Re |
| 36 | Hydroxylated Nar (Eriodictyol) (S) | C₁₅H₁₂O₆ | Or, Re |
| 37 | Hydroxylated Nar-hexose (eriodictyol-hexose) | C₂₁H₂₂O₁₁ | Or |
| 31 | Methyl ether of hydroxylated N or Methyl ether of | C₁₆H₁₄O₆ | Or, Re |
| | hydroxylated NarCh I | | |
| 34 | Methyl ether of hydroxylated N or Methyl ether of | C₁₆H₁₄O₆ | Br, Or, Re |
| | hydroxylated NarCh II | | |
| 32 | Methyl ether of hydroxylated N-hexose, or Methyl ether | C₂₂H₂₄O₁₁ | Or, Re |
| | of hydroxylated NarCh-hexose I | | |
| 33 | Methyl ether of hydroxylated N-hexose, or Methyl ether | C₂₂H₂₄O₁₁ | Or, Re |
| | of hydroxylated NarCh-hexose II | | |
| 64 | Phloretin (S) | C₁₅H₁₄O₅ | Br, Or, Re |
| 65 | Phloretin-di-C-hexose | C₂₇H₃₄O₁₅ | Or, Re |
| 66 | Phloretin-trihexose | C₃₃H₄₄O₂₀ | Or, Re |
| 16 | Quercetin-dihexose-deoxyhexose | C₃₃H₄₀O₂₁ | Or |
| 41 | Hydroxybenzoic acid-hexose | C₁₃H₁₆O₈ | Or |

Metabolites up-regulated in the y mutant peel

| | | | |
|---|---|---|---|
| 63 | Caffeic acid-hexose IV | C₁₅H₁₈O₉ | Re |
| 60 | Ferulic acid-dihexose | C₂₂H₃₀O₁₄ | Or |
| 61 | Ferulic acid-hexose III | C₁₆H₂₀O₉ | Or |
| 57 | N-Feruloylputrescine I | C₁₄H₂₀N₂O₃ | Or |
| 69 | Feruloyltyramine-hexose | C₂₄H₂₉NO₉ | Or |

¹The peak number here corresponds to the numbers given for all metabolites detected by the UPLC-QTOF-MS analysis in this study.
²NarCha—Naringenin Chalcone.
³(S) - compound was identified by comparison of its retention time and mass spectrum with those of the authentic standard.
⁴Nar—Naringenin.
⁵Br, Or and Re are for breaker, orange and red stages of fruit development, respectively.

TABLE 7
UPLC-QTOF-MS detected metabolites that are differentially
produced between y mutant and wt fruit flesh tissues

| | | | |
|---|---|---|---|
| Peak | Putative | Molecular | Differential |
| No.¹ | metabolite | formula | stage |

Metabolites down-regulated in the y mutant flesh

| | | | |
|---|---|---|---|
| 23 | NarCh² (S)³ | C₁₅H₁₂O₅ | Br, Or, Re⁵ |
| 26 | NarCh-hexose II | C₂₁H₂₂O₁₀ | Or, Re |
| 22 | Nar⁴ (S) | C₁₅H₁₂O₅ | Or |
| 29 | Nar-dihexose I | C₂₇H₃₂O₁₅ | Or, Re |
| 65 | Phloretin-di-C-hexose | C₂₇H₃₄O₁₅ | Or, Re |
| 66 | Phloretin-trihexose | C₃₃H₄₄O₂₀ | Or, Re |

Metabolites up-regulated in the y mutant flesh

| | | | |
|---|---|---|---|
| 58 | N-Feruloylputrescine II | C₁₄H₂₀N₂O₃ | Re |
| 69 | FerUloyltyramine-hexose | C₂₄H₂₉NO₉ | Or |
| 70 | Caffeoylputrescine | C₁₃H₁₈N₂O₃ | Re |

¹The peak number here corresponds to the numbers given for all metabolites detected by the UPLC-QTOF-MS analysis in this study.
²NC—Naringenin Chalcone.
³(S) - compound was identified by comparison of its retention time and mass spectrum with those of the authentic standard.
⁴N—Naringenin.
⁵Br, Or and Re are for breaker, orange and red stages of fruit development, respectively.

The two large groups pf flavonoids detected in the peel tissue were NarCh and Nar derivatives, as well as quercetin-derivatives. Apart from a single derivative, all members of the NarCh/Nar group were down-regulated in the y mutant peel, while the levels of most quercetin-derivatives were not altered. Other flavonoids identified in y and wt peels were eriodictyol and one of its derivatives, as well as two kaempferol derivatives, with only eriodictyol and its derivative being down-regulated in the y mutant peel. Additional phenylpropanoid and flavonoids were also detected in the peel, including benzoic acid and two of its derivatives, three coumaric acid derivatives, six caffeic acid derivatives, seven ferulic acid derivatives, phloretin and two of its derivatives, as well as trans-resveratrol. Of these, only one benzoic acid derivative, one coumaric acid derivative, phloretin and its two derivatives, as well as the trans-resveratrol were down-regulated in the y mutant fruit peel. Secondary metabolites that showed enhanced levels in y were 4 ferulic acid derivatives and a single caffeic acid derivative, which are all part of the phenylpropanoid pathway branch associated with lignin metabolism.

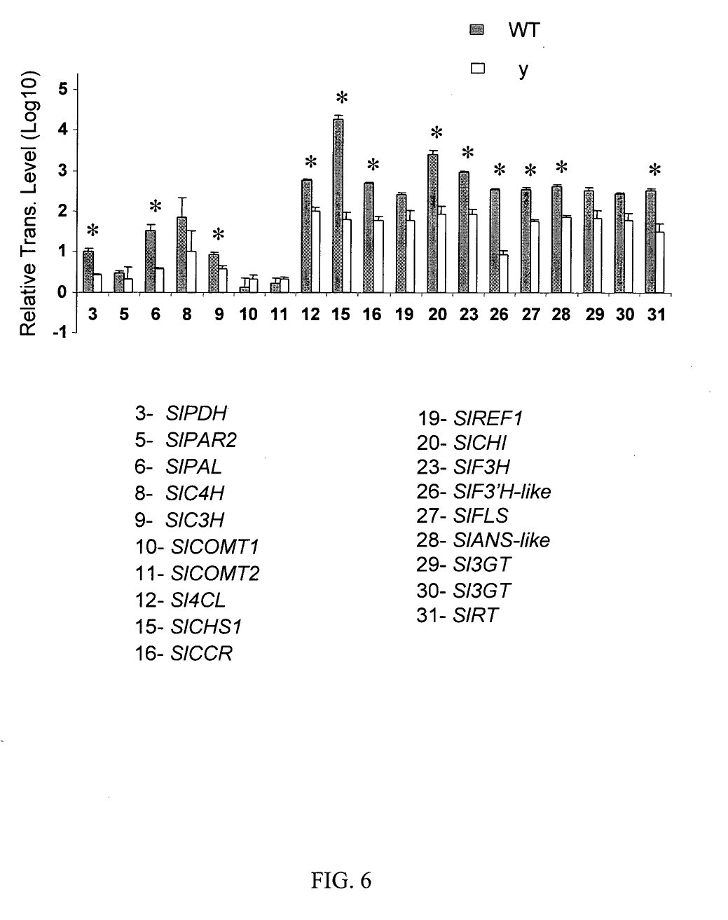

As indicated above, microarray gene expression analysis revealed the down-regulation of twenty-one transcripts putatively associated with the phenylpropanoid/flavonoid pathway in the y mutant peel (FIG. 3A). These included early shikimate pathway and general phenylpropanoid related transcripts (SlPDH and SlCM, as well as Sl4CL and SlCCR, respectively), transcripts corresponding to the flavonoid pathway and associated with NarCh and Nar biosynthesis (e.g. SlCHS, SlCHI and SlF3H), as well as SlFLS transcripts that putatively catalyze the formation of flavonols. The down-regulation of most of these genes was corroborated by the results of Real Time-PCR analysis that included five additional putative phenylpropanoid/flavonoid related genes (SlANS, SlF3′H, two SlCOMT genes and SlREF1) not present on the array (FIG. 6). Expression levels of the two additional genes, SlCOMT and the SlREF1, that are putative structural genes in the lignin metabolism branch and related to the biosynthesis of ferulic acid derivatives, did not differ between the y mutant and wt peels. According to the array results, three putative phenylpropanoid/flavonoid related genes (SlPAR, SlF3H and a tomato acyltransferase) seem to be up-regulated in at least one of the tested stages of fruit development. However, in the case of the SlPAR gene, Real Time-PCR analysis did not confirm the microarray results, and in the case of SlF3H a different putative SlF3H was found to be down-regulated in the y mutant peel by both the array and RT-PCR analyses. Overall, gene expression according to both microarray and Real Time-PCR analyses further corroborated the wide alterations exhibited by the phenylpropanoid/flavonoid pathway in the y mutant peel tissue.

While the levels of phenylpropanoid/flavonoid metabolites were also altered in the y mutant flesh, it was much less pronounced as compared to the changes in the peel tissue. This finding is in accordance with many previous studies showing lower activity of the phenylpropanoid/flavonoid pathway in tomato flesh (e.g. Mintz-Oron et al 2008, ibid; Moco S. et al., 2007. J. Exp. Bot. 58: 4131-4146; Muir et al., 2001, ibid; Bovy et al., 2002 ibid). Nar-Cha, Nar and phloretin and their derivatives were also found to be down-regulated in the y mutant flesh tissue as compared to that of the wt. Noteworthy are phenylalanine and benzoic acid from the upper part of the phenylpropanoid pathway, which showed significant down-regulation only in the flesh of the y mutant. Feruloyltyramine hexose, N-feruloylputrescine and caffeoylputrescine in the lignin-related branch of the phenylpropanoid pathway were up-regulated in both flesh and peel of the y mutant. Although the expression of all structural genes from the phenylpropanoid/flavonoid pathway was overall lower in the flesh than in the peel (of both y and wt fruit), their down-regulation in y (relative to the wt expression levels) was also evident in the flesh (FIG. 7). Thus, extensive alterations in the phenylpropanoid/flavonoid pathway were also detected in the flesh of the y mutant.

### Example 5

### Chromosomal Location of SlMYB12

The broad effects on gene expression and metabolism in y suggested that a gene upstream to SlCHS, possibly a regulatory factor, is responsible for the y mutant phenotype. Microarray analysis revealed down-regulated expression of two members of the R2R3-MYB transcription factor family, SlTHM27 and SlMYB4-like (TC174616 and TC184379, respectively), in y fruit at the breaker and orange stages. Sequencing the coding regions of both transcripts from y and wt fruit cDNA samples revealed single nucleotide polymorphisms (SNPs) that are not expected to alter the function of the putatively translated proteins. Furthermore, these two transcription factors were mapped to chromosomes 10 and 6 and not to the previously known y mutation locus on chromosome 1 (Rick and Butler 1956, ibid).

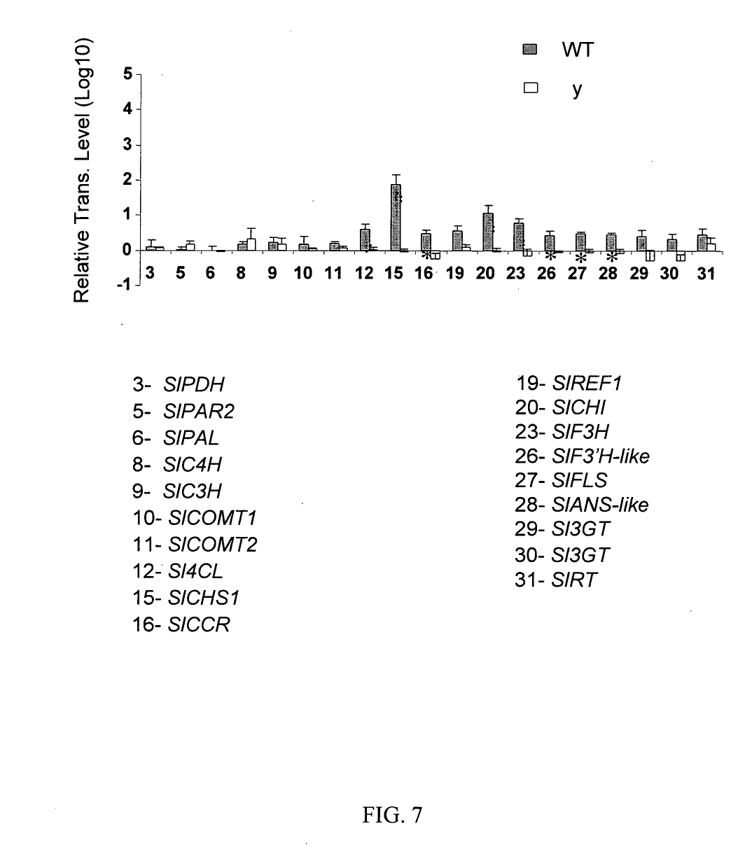

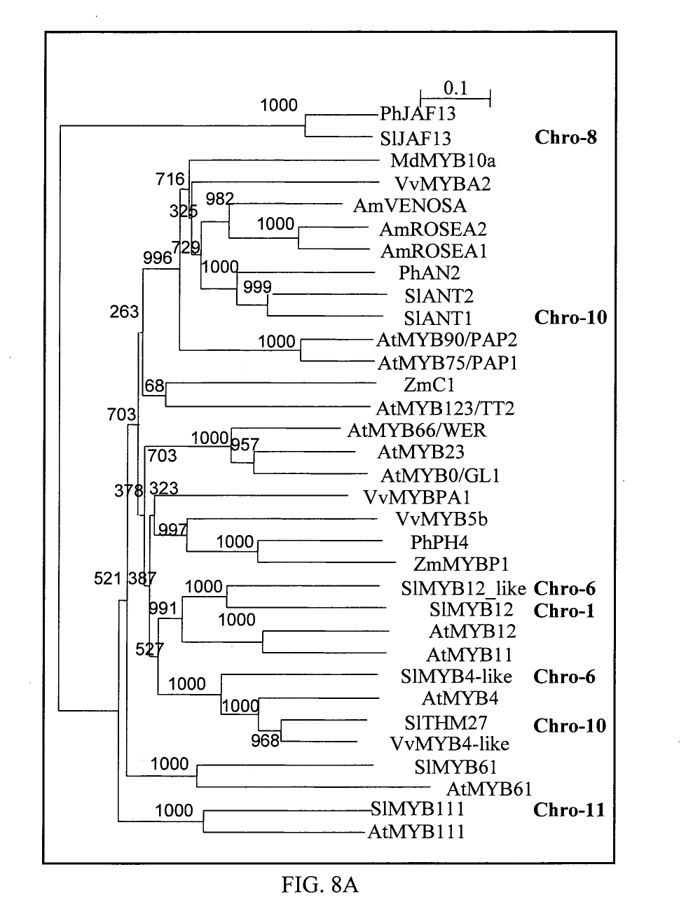

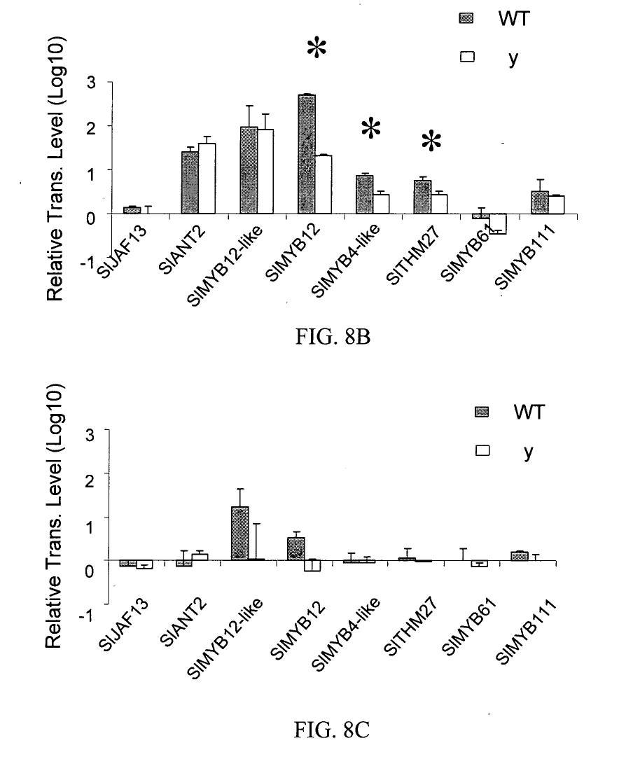

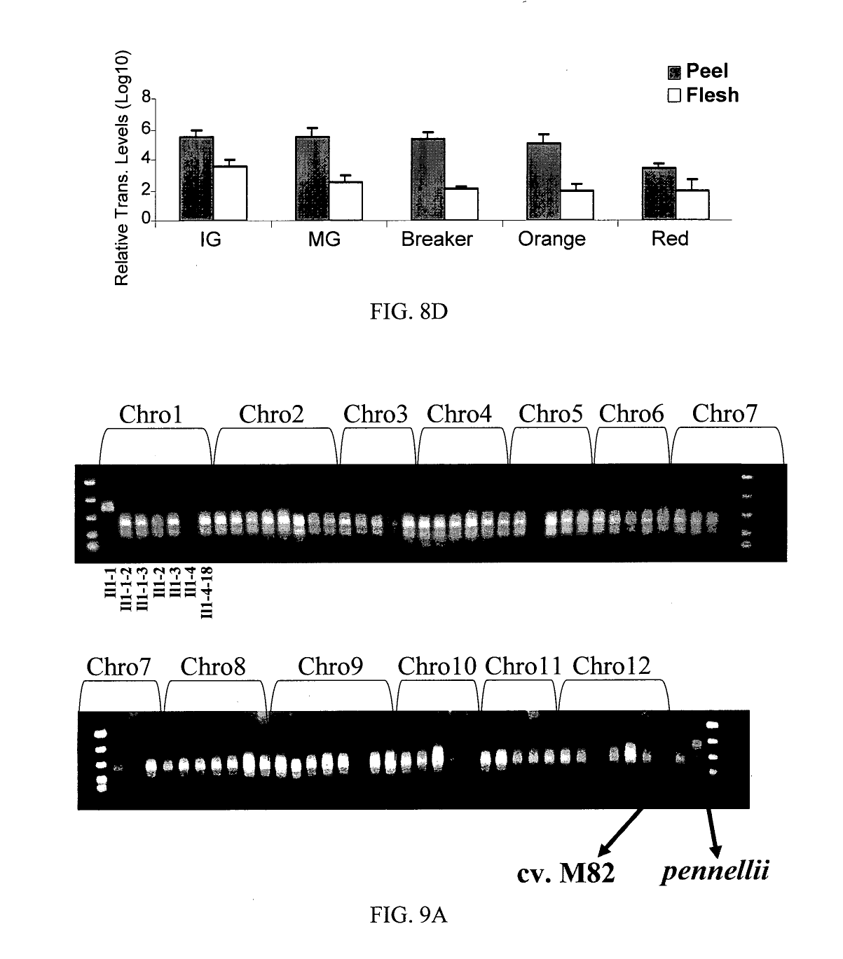

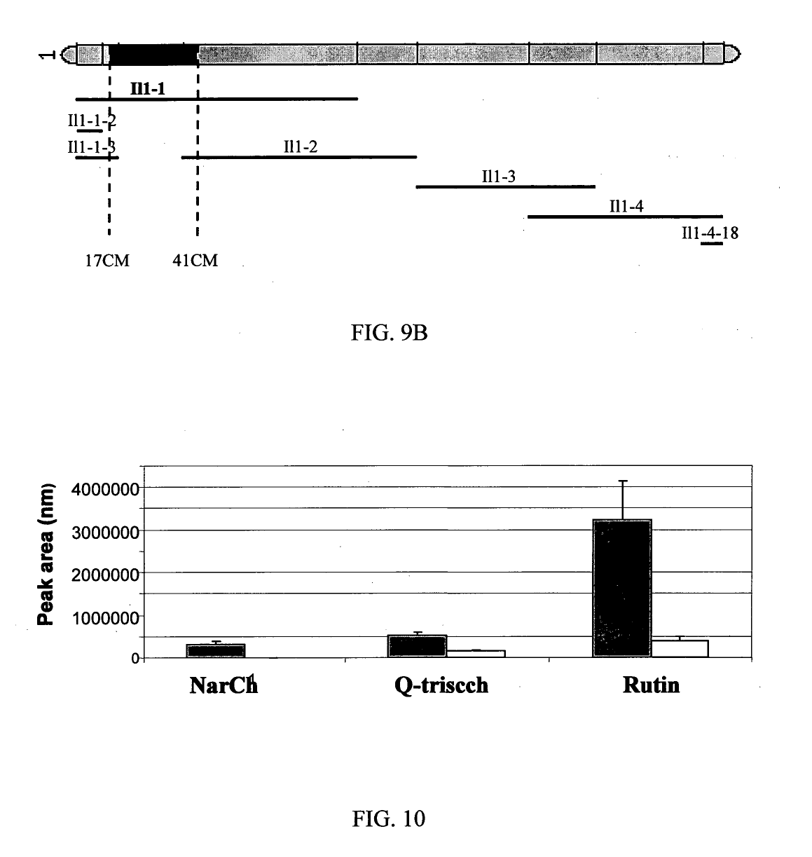

In order to find the regulatory factor that is responsible for the y mutant phenotype, we have identified and reconstructed seven additional putative tomato transcription factors that are orthologs/homologs of known flavonoid-related regulators from other species (six R2R3-MYB family members and one basic-helix-loop-helix (bHLH)). Phylogenetic analysis performed with the predicted protein sequences of these tomato regulators and sequences of known flavonoid related transcription factors from other species (FIG. 8A) revealed three paralogous pairs including; SlMYB12 and SlMYB12-like, SlMYB4-like and SlTHM27, as well as SlANT1 and SlANT2, that are putative orthologs/homologs of the Arabidopsis MYB12 and MYB4 and the petunia AN2 transcription factors, respectively. Two additional tomato R2R3-MYB genes (SlMYB111 and SlMYB61) are orthologs of the Arabidopsis MYB111 and MYB61, and the bHLH transcription factor (SlJAF13) is an ortholog of the petunia JAF13. Sequencing of transcripts corresponding to the seven additional putative regulators from both y and wt did not yield any sequence lesion which is likely to alter the function of their putatively encoded proteins. Expression analysis revealed that only one of these additional regulators (SlMYB12), exhibits altered expression levels in the y mutant fruit tissues (FIG. 8B). We were able to assign a chromosome location to four of these seven candidate regulators; SlMYB12, SlMYB12-like, SlMYB111 and SlJAF13 that were mapped on chromosomes 1, 6, 11 and 8, respectively, while SlANT1 was recently mapped to tomato chromosome 10 (Sapir M. et al., 2008. J. Hered. 99: 292-303). Partial overlaps between the pennellii chromosome segments inserted into the interspecific introgression lines (IL) used for the gene mapping (Eshed Y. and Zamir D. 1995. Genetics 141: 1147-1162) allowed the localization of SlMYB12 to an interval between cM 17 and 41 on chromosome 1, which contains the previously defined y mutation locus, 1-30 (FIG. 9). Analysis of SlMYB12 expression during five stages of fruit development demonstrated peel-associated expression that was maximal at the immature green stage and declined as the fruit developed towards the ripe stage (FIG. 8C).

Thus, no tomato regulatory gene was found to harbor a mutation that is likely to alter the function of its predicted protein. Three out of the nine studied transcription factors (SlTHM27, SlMYB4-like and SlMYB12) are significantly down-regulated in the y mutant fruit peel, but only one of these, the peel-associated SlMYB12, maps to a genomic region on chromosome 1 previously reported to harbor the mutation underlying the y mutant phenotype.

### Example 6

### Additional Allele Co-Segregating with Colorless-Peel

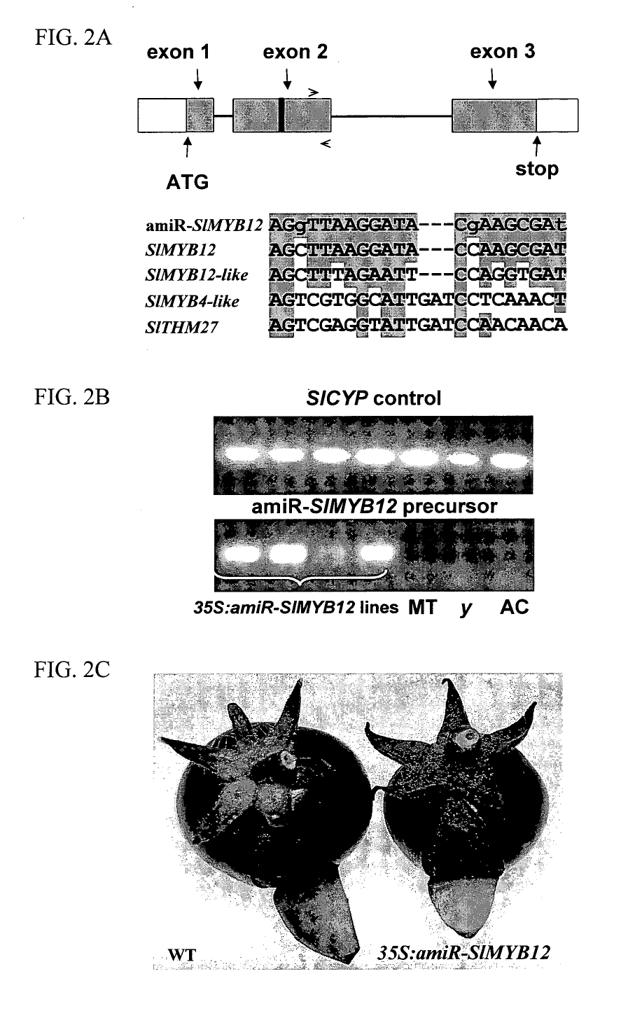

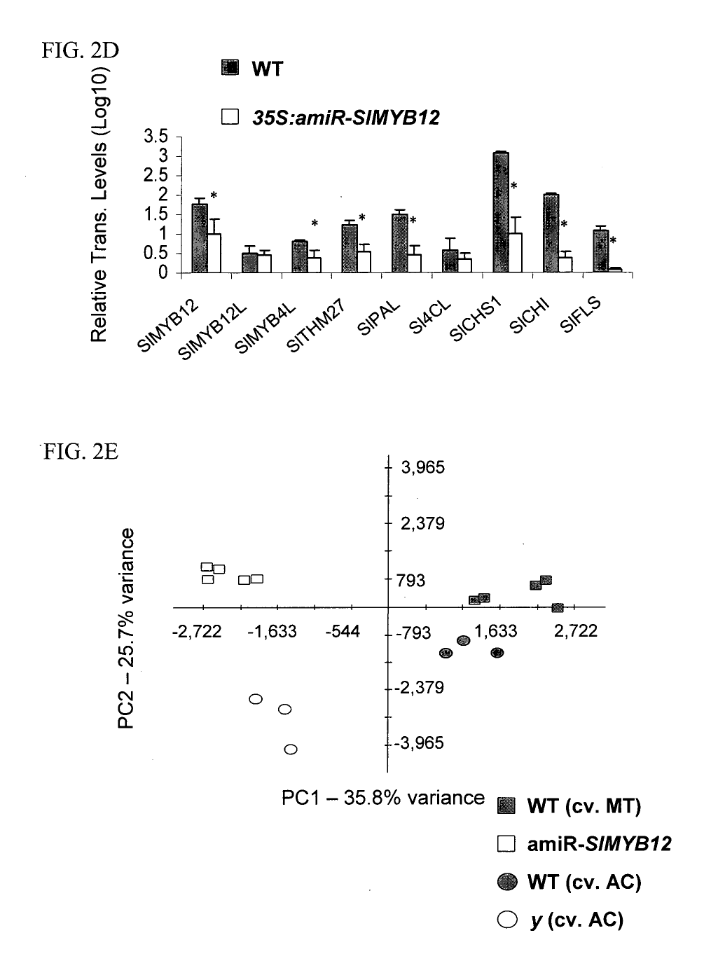

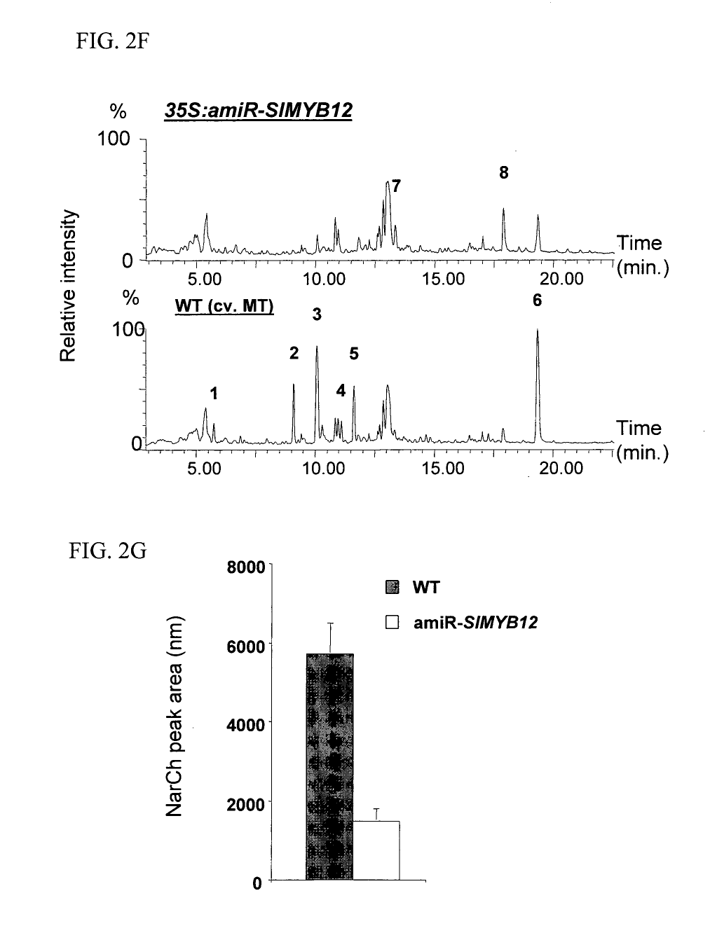

The reconstructed structure of the SlMYB12 gene consists of 1974 bp and includes two introns (FIG. 2A). Random Amplification of cDNA Ends (RACE) analyses revealed two alternative polyadenylated versions at the 3′ UTR of the wt SlMYB12 transcript at positions 73 bp (S version) or 204 bp (L version) downstream from the stop codon (FIG. 2A). Sequencing of the genomic SlMYB12 from the Ailsa Craig (AC) cultivar and the y mutant yielded several SNPs, but none of these were specific to the y genotype. We have also reconstructed and sequenced more than 0.5 kb of the SlMYB12 upstream region. However, no differences between y and wt sequences were detected in the upstream region of the gene.

In addition, the SlMYB12 gene from some other colorless-peel tomato lines derived from different origins was sequenced. Several Introgression Lines (IL), generated by an interspecific cross between a tomato elite processing inbred line (Solanum lycopersicum E6203) and the wild species Solanum neorickii (previously known as L. parviflorum), (LA2133; Fulton T. M. et al., 2000. Theor Appl Genet 100: 1025-1042) were found to carry the same combination of sequence changes in introns and exons of their MYB12 gene. These sequence changes including also some SNPs and a 3 bp deletion, that are expected to cause five missense changes (K227M; R237E; V245A; N256S and T331A) and one amino acid deletion (N315del) in the corresponding protein (FIG. 10). Prediction programs (PSIPRED http://bioinf.cs.ucl.ac.uk/psipred/; JPRED http://www.compbio.dundee.ac.uk/˜www-jpred/index.html) determined that these amino acid alterations are likely to decrease the protein stability, especially the T331A missense change, which is expected to disturb the formation of a helix structure at the C-terminus. Furthermore, 3′ RACE analysis performed on SlMYB12 transcripts from these colorless-peel lines revealed that the multiple sequence changes introduced new signal/s for premature polyadenylation (pad) of the SlMYB12 transcripts, at amino acids 237, 264, 273, 320 and 327 within the coding sequence of exon 3 (pad1a, pad2a, pad3a, pad4a, pad5a) or just on the stop codon (pad6a) (FIG. 2A and FIG. 9B). The integrated sequence changes comprising this putative y allele (termed y-1) were not detected in the genomic SlMYB12 isolated from the M82, MicroTom (MT), AC and E6203 cultivars as well as in the y mutant characterized in this study, and were found to co-segregate with the colorless-peel phenotype among more than 100 lines of the examined IL population.

### Example 7

### An Artificial MicroRNA Targeting SlMYB12 Induces a y-Like Phenotype in Transgenic Plants

In order to confirm the implication of SlMYB12 as the regulator underlying the y phenotype, an artificial microRNA that specifically targets SlMYB12 (amiR-SlMYB12) was designed. The amiR-SlMYB12 was expressed it in tomato (cv. MT) under the control of the constitutive CaMV 35S promoter (FIG. 2B). Fruit derived from four transgenic plants exhibited the typical y mutant colorless-peel phenotype (FIG. 2C). Real-Time PCR analysis revealed significant down-regulation of SlMYB12 as well as of the two additional transcription factors, SlTHM27 and SlMYB4-like. As described above, these three factors were also down-regulated in the y mutant. Significantly reduced expression levels were also detected for several tested phenylpropanoid/flavonoid-related structural genes, including SlPAL, SlCHS, SlCHI, and SlFLS (FIG. 2D). Expression of SlMYB12-like, the closest paralogue of SlMYB12, was not altered in the amiR-SlMYB12 expressing plants.

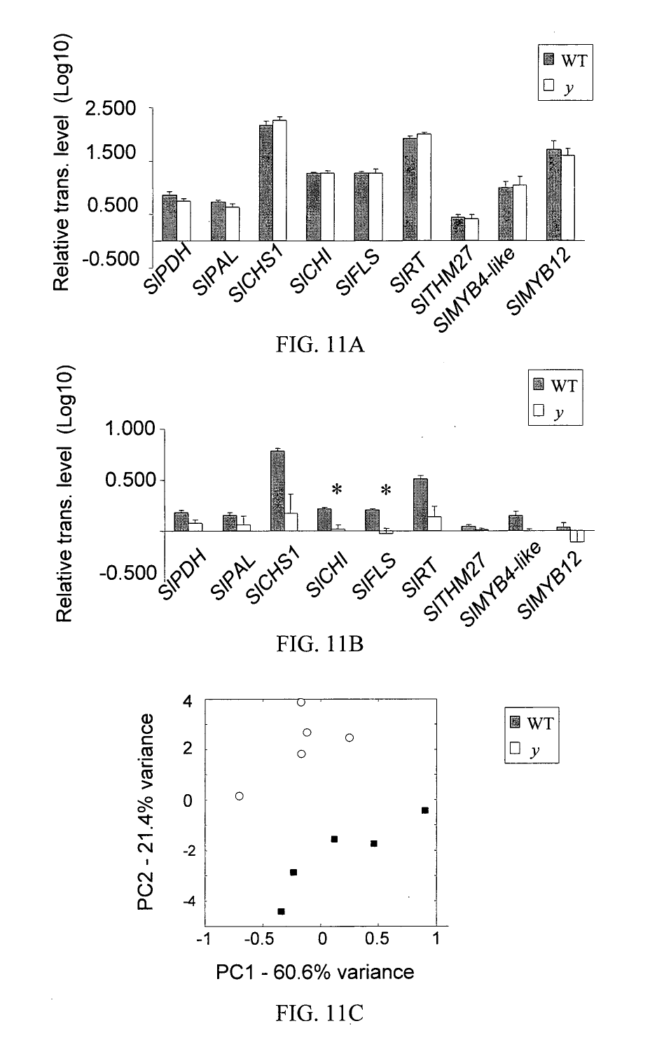

PCA analysis of metabolic profiling data (LC-MS) obtained from peel samples of ripe fruit of the amiR-SlMYB12 and its wt (cv. MT), as well as of the y mutant and its wt (cv. AC) could clearly distinguish between the profiles of amiR-SlMYB12 and wt cv. MT (FIG. 2E). The metabolite profiles of wt peels from cv. MT and cv. AC differed as well, but were much closer to each other than to the y mutant or to the amiR-SlMYB12 profiles. A significant down-regulation in levels of several flavonoids including NarCh, phloretin di-hexoside and several flavonol-conjugates, as well as up-regulation in levels of a few caffeic acid derivatives in the amiR-SlMYB12 expressing plants were detected. (FIGS. 2F and 11). The putative identity of the differential compounds in FIG. 2F is: 1-quercetin-dihexose-deoxyhexose, 2-quercetinhexose-deoxyhexose-pentose, 3-quercetin rutinoside (Rutin), 4-phloretin-di-C-hexose, 5-kaempferol-glucose-rhamnose, 6-naringenin chalcone, 7-dicaffeoylquinic acid III, 8-tricaffeoylquinic acid. Upper and lower panel indicate metabolites that showed elevated or reduced levels in the transgene samples in comparison to those of their corresponding wt, respectively.

### Example 8

### Phenotype Complementation was Driven by Constitutive Expression of SlMYB12 on y Genetic Background

In preliminary experiments, expression of SlMYB12 (driven by the 35S CaMV promoter) on the y mutant background, resulted in fruit displaying partial complementation of the y phenotype. UPLC-PDA (photo diode array) analysis of red fruit peels revealed significantly different levels of flavonoids (including NarCh, quercetin-hexose-deoxyhexose-pentose and quercetin rutinoside) between phenotype complementation regions (in peels of 35S:SlMYB12) and y mutant peels (FIG. 10).

### Example 9

### Metabolic Alterations in Organs Other than Fruit in the y Mutant

To examine whether the lesion underlying the y phenotype effects metabolism in plant organs other than the fruit, we evaluated the expression of 6 structural and 3 regulatory phenylpropanoid/flavonoid associated genes in leaves of y and wt plants. While in young leaves the expression of these genes was not different between y and wt (FIG. 11A), a clear trend of down-regulated expression in y was evident for all the tested genes in fully expended leaves. This down-regulation was highly significant in the case of SlCHI and SlFLS (FIG. 11B). In addition, PCA analysis of metabolite data sets obtained by UPLC-QTOF-MS profiling of roots derived from y and wt seedlings clearly distinguished between the profiles of the two genotypes (FIG. 11C). Thus, the effects of the lesion underlying the y mutant phenotype are not restricted to fruit tissues.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An isolated polynucleotide encoding a SlMYB12 variant transcript, the polynucleotide comprising at least one alteration compared to the wild type SlMYB12, the wild type having the nucleic acid sequence set forth in SEQ ID NO:1, wherein the variant transcript results in down regulated expression and/or activity of the encoded SlMYB12 protein.

2. The isolated polynucleotide of claim 1, comprising at least one alteration in any one of the promoter region, intron 1, intron 2, exon 3 or combinations thereof compared to the wild type SlMYB12 having the nucleic acid sequence set forth in SEQ ID NO:1.

3. The isolated polynucleotide of claim 2, comprising a nucleic acid sequence as set forth in any one of SEQ ID NOs: 2-28.

4. The isolated polynucleotide of claim 2, wherein the alterations are present within positions 905-994 or positions 1474-1728 of SEQ ID NO:1.

5. The isolated polynucleotide of claim 4, comprising a nucleic acid sequence as set forth in any one of SEQ ID NO:29 and SEQ ID NO:30.

6. (canceled)

7. (canceled)

8. The isolated polynucleotide of claim 1, having a nucleic acid sequence as set forth in SEQ ID NO:31.

9. A detecting agent capable of detecting the polynucleotide of claim 1.

10. (canceled)

11. The detecting agent of claim 9, wherein said detecting agent differentially hybridizes to said polynucleotide compared to a wild-type SlMYB12 polynucleotide having the nucleic acid sequence set forth in SEQ ID NO:1.

12. The detecting agent of claim 11, wherein said polynucleotide has a nucleic acid sequence as set forth in any one of SEQ ID NOs:2-31 or part thereof.

13. The detecting agent of claim 9, wherein said detecting agent is a primer pair capable of selectively amplifying said polynucleotide compared to a wild type SlMYB12 having the nucleic acid sequence as set forth in SEQ ID NO:1.

14. The detecting agent of claim 13, wherein said polynucleotide has a nucleic acid sequence as set forth in any one of SEQ ID NOs:2-31 or part thereof.

15. A method of screening for genetic markers indicative of the colorless peel y mutant phenotype in a tomato plant, comprising: (a) comparing the genomic polynucleotide sequence of the SlMYB12 gene having SEQ ID NO:1 of the wild type tomato plant or a fragment thereof to the genomic polynucleotide sequence of a SlMYB12 gene in a tissue sample obtained from a y phenotype tomato plant; (b) identifying alterations in the SlMYB12 genomic sequence of the y mutant phenotype, wherein the alterations predict modification in the gene transcription and/or translation; and wherein said alterations are genetic markers indicative of the y mutant phenotype.

16. The method of claim 15, wherein the alteration is identified within a non-coding region selected from the group consisting of an intron and an upstream promoter region.

17. (canceled)

18. The method of claim 15, wherein the sequence alterations result in down regulation of the expression or activity of SlMYB12.

19. A method for identifying a tomato plant capable of producing fruit having the colorless peel y phenotype comprising: (a) providing a sample comprising genetic material from the plant before fruit are produced; (b) determining, in the sample, the sequence of the SlMYB12 gene or its transcript or a part thereof; (c) comparing the sequence to the sequence of a tomato wild type SlMYB12 gene or transcript; and (d) detecting at least one alteration in the SlMYB12 sequence from said sample compared to the wild type, wherein the alteration is indicative of the capability of the plant to produce fruit having a y phenotype.

20. The method of claim 19, wherein the wild type SlMYB12 gene comprises a nucleic acid sequence as set forth in SEQ ID NO:1 and the wild type SlMYB12 transcript comprises a nucleic acid sequence as set forth in SEQ ID NO:32.

21. (canceled)

22. The method of claim 19, wherein the at least one alteration results in down regulation of the expression or activity of SlMYB12.

23. The method of claim 22, wherein the alterations are present within the promoter region, intron 1, intron 2, exon 3 or combinations thereof compared to the wild type SlMYB12, and wherein said alterations predict modification in the gene transcription and/or translation.

24. The method of claim 23, wherein the SlMYB12 gene of the sample comprises a nucleic acid sequence as set forth in any one of SEQ ID NOs: 2-31.

25. (canceled)

26. An isolated polynucleotide encoding a wild type SlMYB12 polypeptide having SEQ ID NO:33.

27. The isolated polynucleotide of claim 26, comprising a nucleic acid sequence as set forth in any one of SEQ ID NO:1 and SEQ ID NO:32.

28. (canceled)

29. A transgenic plant comprising at least one cell comprising in its genome an exogenous polynucleotide according to claim 26, wherein the plant has an elevated content of at least one phenylpropanoid selected from the group consisting of flavonoids, chlorogenic acid and derivatives thereof compared to a non-transgenic plant.

30. The transgenic plant of claim 29, wherein the polynucleotide comprises a nucleic acid sequence as set forth in any one of SEQ ID NO:1 and SEQ ID NO:32.

31. (canceled)

32. The transgenic plant of claim 29 having elevated content of at least one of naringenin, naringenin chalcone, eridictyol, phloretin, resveratrol, Quercetin-hexose-deoxyhexose-pentose (Q-triscch) and Quercetin rutinoside (Rutin) compared to a non-transgenic plant.
